(19) United States
(12) Patent Application Publication
(10) Pub. No.: US 2012/0288857 A1
(43) Pub. Date: Nov. 15, 2012

(54) MULTIFUNCTIONAL PROBE-PRIMERS

(75) Inventor: Kenneth J. Livak, San Jose, CA (US)

(73) Assignee: Fluidigm Corporation, South San Francisco, CA (US)

(21) Appl. No.: 13/366,178

(22) Filed: Feb. 3, 2012

Related U.S. Application Data
(60) Provisional application No. 61/439,350, filed on Feb. 3, 2011.

(51) Int. Cl. C07H 21/00; G01N 21/64; C12Q 1/68

(52) U.S. Cl. 435/6.11; 536/24.33; 435/6.12; 536/23.1

(57) ABSTRACT

Methods and reagents for detection and analysis of nucleic acids are provided. Certain methods involves an encoding amplification in which a target sequence is associated with probe-binding sequences and optionally with indexing sequences, (2) an optional distribution step in which the product of the encoding amplification is split into multiple aliquots, and (3) a decoding and detection step in which the presence, absence, quantity, or relative amount of the target sequence in the aliquots is determined. The detection step makes use of a multifunctional “self-digesting” molecular probe comprising a primer polynucleotide and a probe oligonucleotide, linked in a 5′-5′ orientation.

### CROSS-REFERENCE TO OTHER APPLICATIONS

This application claims priority to U.S. Provisional Application No. 61/439,350, filed Feb. 3, 2011, the entire content of which is incorporated herein by reference.

### FIELD OF THE INVENTION

The invention related to genetic assays using molecular probes, and methods and reagents for carrying out such assays.

### BACKGROUND

The high cost of fluorogenic probes used in quantitative PCR (qPCR) assays has prompted schemes that use a “Universal Probe”. A Universal Probe, sometimes called a “Universal Template Probe,” is a generic fluorogenic probe that can be used to generate signal for any and all quantitative PCR (qPCR) assays. One of the advantages of this method is that different target DNA sequences can be detected employing the same labeled probe, which substantially reduces the cost of real-time PCR set-up. One example, described by Zhang et al., 2003, Nucl. Acids Res. 31:e123, is an approximately 20 base attachment to the 5′ end of a PCR primer, able to hybridize to a complementary TaqMan™ probe. U.S. Pat. Nos. 7,153,658 and 7,601,821 describe variations of this scheme by adding a generic set of primers to the Universal Probe. In this case, a multiplex ligation reaction is performed to associate each specific target segment with its own pair of encoding PCR primers.

### BRIEF DESCRIPTION OF THE INVENTION

In various aspects, the invention includes, but is not limited to, the following embodiments:

### Embodiment 1

A multifunctional molecular probe comprising a) a first oligonucleotide that is a primer with an extendible 3′ terminus, b) a second oligonucleotide, comprising a first signal moiety, wherein the first oligonucleotide and the second oligonucleotide are connected by a linker in a 5′-5′ orientation.

### Embodiment 2

The multifunctional molecular probe of Embodiment 1 wherein the 5′ terminus of the first oligonucleotide is connected by a linker to the 5′ terminus of the second oligonucleotide.

### Embodiment 3

The multifunctional molecular probe of Embodiment 1 wherein the first signal moiety comprises a first fluorophore and the first oligonucleotide does not comprise a signal moiety.

### Embodiment 4

The multifunctional molecular probe of Embodiment 3 wherein the second oligonucleotide comprises a second fluorophore, and the first and second fluorophores comprise a first donor-acceptor pair.

### Embodiment 5

The multifunctional molecular probe of Embodiment 1 comprising a third oligonucleotide, wherein the first oligonucleotide and the third oligonucleotide are connected by a linker in a 5′-5′ orientation, wherein the third oligonucleotide comprises a second signal moiety, which is different from the first signal moiety wherein the first and second oligonucleotides and the first and third oligonucleotides may be linked to the first oligonucleotide by the same linker molecule or a different linker molecule.

### Embodiment 6

The multifunctional molecular probe of Embodiment 1 wherein the 5′ terminus of the third oligonucleotide is connected by a linker to the 5′ terminus of the first oligonucleotide.

### Embodiment 7

The multifunctional probe of Embodiment 5, wherein the second signal moiety comprises a fluorophore.

### Embodiment 8

The multifunctional probe of Embodiment 7, wherein the second signal moiety comprises a second donor-acceptor pair.

### Embodiment 9

The multifunctional molecular probe of Embodiment 7 wherein the second and third oligonucleotides each comprise a fluorophore, and the first oligonucleotide does not.

### Embodiment 10

The multifunctional molecular probe of Embodiments 1-10 wherein the second oligonucleotide comprises a donor fluorophore at or close to the 5′ terminus and an acceptor fluorophore at or close to the 3′ terminus, and the second oligonucleotide comprises a partially self-complementary sequence such that the oligonucleotide may adopt a stem-and-loop structure in which the donor and acceptor are in close proximity or a linear structure in which the donor and acceptor are not in close proximity.

### Embodiment 11

The multifunctional molecular probe of Embodiments 5-9 wherein the third oligonucleotide comprises a donor fluorophore at or close to the 5′ terminus and an acceptor fluorophore at or close to the 3′ terminus, and the third oligonucleotide comprises a partially self-complementary sequence such that the oligonucleotide may adopt a stem-and-loop structure in which the donor and acceptor are in close proximity or a linear structure in which the donor and acceptor are not in close proximity.

### Embodiment 12

The multifunctional molecular probe of any preceding Embodiment wherein the linker comprises HEG, TEG, PEG, glycerol, 1′2′-dideoxyribose, or C2 alkyl-C15 alkyl.

### Embodiment 13

The multifunctional molecular probe of any preceding Embodiment wherein the donor is selected from the group consisting of a xanthene dye, a cyanine dye, and a dansyl derivative.

### Embodiment 14

A molecular construct comprising two polynucleotides and a non-nucleotide linker, wherein the first polynucleotide is an oligonucleotide comprising a signal moiety and comprising a sequence π; the second polynucleotide comprises a target sequence and a probe binding sequence P, the target sequence being 5′ to P; wherein π and P are sufficiently complementary to each other to hybridize and form a double stranded polynucleotide segment; and wherein the linker links first and second polynucleotides in a 5′-5′ orientation.

### Embodiment 15

The molecular construct of Embodiment 14, wherein π and P are exactly complementary.

### Embodiment 16

The molecular construct of Embodiment 14 or 15, wherein the signal moiety comprises a fluorophore.

### Embodiment 17

The molecular construct of Embodiment 16 wherein signal moiety comprises a donor-acceptor pair.

### Embodiment 18

The molecular construct of Embodiments 14-17 wherein the oligonucleotide comprises a partially self-complementary sequence such that the oligonucleotide may adopt a stem-and-loop structure.

### Embodiment 19

The molecular construct of Embodiments 14-18 wherein the second polynucleotide comprises an indexing sequence, and said indexing sequence is 5′ to, or is 3′ to, probe binding sequence P.

### Embodiment 20

The molecular construct of Embodiment 19 wherein the indexing sequence is 5′ to, and optionally contiguous with, P.

### Embodiment 21

The molecular construct of Embodiment 19 wherein the indexing sequence is 3′ to, and optionally contiguous with, P.

### Embodiment 22

A pair of molecular constructs, each comprising three linked polynucleotides, wherein 1) the first molecular construct comprises a first polynucleotide that is an oligonucleotide comprising a sequence π₁ and a first signal moiety; a second polynucleotide that is an oligonucleotide comprising a sequence π₂ and a second signal moiety; a third polynucleotide that comprises a first target sequence, and a probe binding sequence P₁, the target sequence being 5′ to P₁; 2) the second molecular constructs comprises a second polynucleotide that is an oligonucleotide comprising a sequence π₁ and the first signal moiety; a second polynucleotide that is an oligonucleotide comprising a sequence π₂ and the second signal moiety; a third polynucleotide that comprises a second target sequence, and a probe binding sequence P₂, the target sequence being 5′ to P₂; wherein in the first molecular construct, P₁ is sufficiently complementary to π₁ to hybridize and form a double stranded polynucleotide segment; and wherein in the second molecular construct, P₂ is sufficiently complementary to π₂ to hybridize and form a double stranded polynucleotide segment; and wherein the first and second target sequences are different wherein the first and second signal moieties are different wherein probe binding sequences P₁ and P₂ are different; and wherein in each construct a linker links the first polynucleotide and the third polynucleotide in a 5′-5′ orientation and the same or a different linker links the first polynucleotide and the third polynucleotide in a 5′-5′ orientation.

### Embodiment 23

The pair of molecular constructs of Embodiment 22, wherein π₁ and P₁ are exactly complementary and/or wherein π₂ and P₂ are exactly complementary.

### Embodiment 24

The pair of molecular constructs of Embodiment 22 or 23, wherein the first and/or second signal moieties comprises a fluorophore.

### Embodiment 25

The pair of molecular constructs of Embodiment 24 wherein at least one signal moiety comprises a donor-acceptor pair.

### Embodiment 26

The pair of molecular constructs of Embodiment 22 wherein a single linker molecule links the first, second and third polynucleotides.

### Embodiment 27

The pair of molecular constructs of Embodiment 22, wherein in one or both constructs in a pair, at least one of the first and second polynucleotides comprises a self-complementary (stem-loop forming) sequence.

### Embodiment 28

The pair of molecular constructs of Embodiment 27 wherein the first and second polynucleotides of both constructs comprise self-complementary (stem-loop forming) sequences.

### Embodiment 29

A composition comprising a molecular construct of Embodiment 19-21 and an oligonucleotide primer comprising a sequence complementary to the indexing sequence.

### Embodiment 30

A composition comprising a pair of molecular constructs of 22-28, wherein the third polynucleotides of each construct comprise an indexing sequence 3′ to, and optionally contiguous with, P, each comprises an oligonucleotide primer complementary to and annealed to the indexing sequence, and the indexing sequences of the two constructs are different.

### Embodiment 31

The composition of Embodiment 14 in which the linker links the 5′ nucleotide of the first polynucleotide to the 5′ nucleotide of the second polynucleotide.

### Embodiment 32

A detection method comprising i) combining a) a molecular construct according to Embodiment 14-19 and 21, wherein the second polynucleotide comprises an indexing sequence I, and said indexing sequence is 3′ to, and optionally contiguous with, P; b) an oligonucleotide primer comprising a sequence complementary to the indexing sequence; and c) a DNA polymerase; and ii) maintaining the combination under conditions in which the oligonucleotide primer is extended using the second polynucleotide as a template to produce an extension product, and the extension results in cleavage of the first polynucleotide and release of a fluorophore from the first polynucleotide; and iii) detecting the release of the fluorophore.

### Embodiment 33

The detection method of Embodiment 32 wherein the released fluorophore is a donor fluorophore.

### Embodiment 34

The detection method of Embodiment 32 wherein the released fluorophore is an acceptor fluorophore.

### Embodiment 35

A detection method comprising: i) combining a) a molecular construct according to Embodiment 14-17 or 19, wherein the second polynucleotide comprises an indexing sequence I, and said indexing sequence is 3′ to, and optionally contiguous with, P; b) an oligonucleotide primer comprising a sequence complementary to the indexing sequence; and c) a DNA polymerase; and ii) maintaining the combination under conditions in which the oligonucleotide primer is extended using the second polynucleotide as a template to produce an extension product, and the extension results in cleavage of the first polynucleotide and release of a fluorophore from the first polynucleotide; and iii) detecting the release of the fluorophore.

Embodiment 35a. A detection method comprising: i) providing a molecular construct according to claim 18, wherein the second polynucleotide comprises an indexing sequence I, and said indexing sequence is 5′ to, and optionally contiguous with, P; and ii) maintaining the construct under conditions in which sequence π and sequence P hybridize and form a double stranded polynucleotide segment; whereby the second oligonucleotide adopts a linear structure in which the donor and acceptor are not in close proximity and produce a signal; and iii) detecting the signal.

### Embodiment 36

A detection method comprising i) amplifying a target sequence to produce a linear double-stranded amplicon, using primers comprising a first indexing sequence or its complement and a second indexing sequence or its complement, whereby the amplicon comprises the first indexing sequence at one end and the second indexing sequence at the other end; ii) denaturing the double-stranded amplicon and carrying out two or more rounds of amplification, using as primers 1) a multifunctional molecular probe comprising a) a first oligonucleotide that is a primer with an extendible 3′ terminus, wherein the primer comprises the first indexing sequence and a probe binding sequence P positioned 5′ to the first indexing sequence; and b) a second oligonucleotide, comprising a sequence π comprising a donor-acceptor pair comprising a first member at or close to the 5′ terminus of the second oligonucleotide and an second member at or close to the 3′ terminus of the oligonucleotide; and comprising a partially self-complementary sequence such that the oligonucleotide may adopt a stem-and-loop structure in which the donor and acceptor are in close proximity or a linear structure in which the donor and acceptor are not in close proximity; wherein the first oligonucleotide and the second oligonucleotide are connected by a linker in a 5′-5′ orientation; and 2) a primer comprising a) the second indexing sequence and b) a probe binding sequence P positioned 5′ to the second indexing sequence; wherein the amplification rounds produce a molecular construct of Embodiment 14; maintaining said molecular construct under conditions in which sequence π and sequence P hybridize and form a double stranded polynucleotide segment; whereby the second oligonucleotide adopts a linear structure in which the donor and acceptor are not in close proximity and produce a signal; iii) detecting the signal.

### Embodiment 37

The method of Embodiment 36 wherein the 5′ terminus of the first oligonucleotide is connected by a linker to the 5′ terminus of the second oligonucleotide.

### Embodiment 38

A detection method comprising i) amplifying a target sequence to produce a linear double-stranded amplicon, using primers comprising a first indexing sequence or its complement and a second indexing sequence or its complement, whereby the amplicon comprises the first indexing sequence at one end and the second indexing sequence at the other end; ii) denaturing the double-stranded amplicon and carrying out two or more rounds of amplification, using as primers 1) a multifunctional molecular probe comprising a) a first oligonucleotide that is a primer with an extendible 3′ terminus, wherein the primer comprises the first indexing sequence; and b) a second oligonucleotide, comprising a sequence π comprising a donor-acceptor pair comprising a donor fluorophore at or close to the 5′ terminus of the second oligonucleotide and an acceptor fluorophore at or close to the 3′ terminus of the oligonucleotide; and wherein the first oligonucleotide and the second oligonucleotide are connected by a linker in a 5′-5′ orientation; and a primer comprising the second indexing sequence, wherein the amplification rounds produce a molecular construct of Embodiment 14 comprising a first polynucleotide comprising a signal moiety and comprising a sequence π and a second polynucleotide comprising a target sequence and a probe binding sequence P, the target sequence being 5′ to P; iii) maintaining said molecular construct in the presence of (a) an oligonucleotide primer comprising the sequence of the second indexing sequence and (b) DNA polymerase under conditions in which sequence π and sequence P hybridize and form a double stranded polynucleotide segment; the oligonucleotide primer hybridizes to the second indexing sequence and is extended by the DNA polymerase using the second polynucleotide as a template to produce an extension product, and the extension results in cleavage of the first polynucleotide and release of a fluorophore from the first polynucleotide; and iii) detecting the release of the fluorophore.

### Embodiment 39

A multiplex detection method comprising amplifying a first target sequence to provide a double-stranded construct having the structure 5′-I₃-target sequence-P₁-I₁′ and amplifying a second target sequence to provide a double-stranded construct having the structure 5′-I₃-target sequence-P₂-I₂′, wherein I₁′, I₂′, I₃, P₁ and P₂ are polynucleotide sequences from 10 to 70 nucleotides in length; amplifying both constructs in the presence of a probe-primer of any of Embodiments 5-9 and 11, wherein the first oligonucleotide of the probe-primer is complementary to I₃, the third oligonucleotide of the probe-primer is complementary to P₁ and the second oligonucleotide of the probe-primer is complementary to P₂, if the first target is present, a first extension primer comprising a sequence complementary to I₁′, and if the second target is present, a second extension primer comprising a sequence complementary to I₂′; under conditions in which the first extension primer is extended and the extension results in cleavage of the second oligonucleotide sequence and/or release of a fluorophore from the second oligonucleotide and/or a change in configuration of the second oligonucleotide, and the second primer is extended and the extension results in cleavage of the third oligonucleotide sequence and/or release of a fluorophore from the third oligonucleotide and/or a change in configuration of the third oligonucleotide and detecting a cleavage event, a release of a fluorophore, or change in configuration.

### Embodiment 40

The detection method of any of Embodiments 38-39 wherein a cleavage event that results in release of a fluorophore is detected.

### Embodiment 41

The detection method of any of Embodiments 32-33, wherein multiple rounds of denaturation, annealing and extension are carried out.

### Embodiment 42

The detection method of Embodiment 41 wherein at least 5, at least 10, at least 15, or at least 20 rounds are carried out.

### Embodiment 43

The detection method of embodiment 39 wherein I₁′, I₂′, I₃, P₁ and P₂ are polynucleotide sequences from 10 to 70 nucleotides in length or from 14 to 70 nucleotides in length.

### Embodiment 44

The detection method of embodiment 39 wherein the donor-acceptor pair comprises a donor fluorophore at or close to the 5′ terminus of the second oligonucleotide and an acceptor fluorophore at or close to the 3′ terminus of the oligonucleotide.

### Embodiment 45

The detection method of any of embodiments 32-44 wherein there is a distribution step between an encoding step and a detection step.

### Embodiment 46

The detection method of embodiment 45 wherein the distribution step comprises partitioning a sample comprising the encoded target sequences.

### Embodiment 47

The detection method of embodiment 46 wherein the partitioning comprises droplet formation and/or is carried out in a microfluidic channel.

### Embodiment 48

A kit comprising a multifunctional molecular probe of any of Embodiments 1-13, and one or more amplification primers (e.g., PCR or LCR primers) comprising a sequence complementary to the amplicon-binding sequence of said probe

In one aspect the invention provides a molecular probe comprising (a) a first oligonucleotide comprising a signal moiety, (b) a second oligonucleotide that is a primer with an extendible 3′ terminus, wherein the 5′-3′ orientation of the first oligonucleotides is opposite that of the second oligonucleotide. In an embodiment, the first oligonucleotide comprises a fluorescent dye and the second oligonucleotide does not.

In one aspect the invention provides a molecular probe comprising (a) a first oligonucleotide comprising a signal moiety, (b) a second oligonucleotide, and (c) a third oligonucleotide that is a primer with an extendible 3′ terminus, wherein the 5′-3′ orientation of the first and second oligonucleotides is opposite that of the third oligonucleotide. In an embodiment, the first and second oligonucleotides comprise a fluorescent dye and the third oligonucleotide does not.

In one aspect the invention provides a molecular probe comprising (a) a first oligonucleotide, comprising a first reporter dye and a first quencher dye; (b) a second oligonucleotide, comprising a second reporter dye and a second quencher dye; wherein the first reporter dye is not the same as the second reporter dye and/or first quencher dye is not the same as the second quencher dye, and (c) a third oligonucleotide, wherein the first and third oligonucleotides are connected by a non-nucleotide linker in a 5- to 5′ orientation and wherein the first and second oligonucleotides are connected by a non-nucleotide linker or linkers to the third oligonucleotide, the first and third oligonucleotides are oriented 5- to 5′ relative to each other and the second and third oligonucleotides are oriented 5- to 5′ relative to each other. In an embodiment, the third oligonucleotide does not comprise a reporter dye or a quencher dye. In an embodiment, the reporter dye is positioned 5′ to the quencher dye in each of the first and second oligonucleotides. In an embodiment, each of the first and second oligonucleotide comprise a self-complementary (stem-loop forming) sequence between the reporter and quencher dyes. In an embodiment, neither of the first and second oligonucleotide comprise a self-complementary (stem-loop forming) sequence between the reporter and quencher dyes.

In one aspect the invention provides a detection method comprising combining (i) a molecular probe described herein, (ii) a polynucleotide comprising a first sequence complementary to the primer or a portion thereof, a second sequence complementary to the first oligonucleotide or a portion thereof, and a third sequence, (iii) an oligonucleotide primer that binds the complement of the third sequence (iv) a DNA polymerase, under conditions in which the primer is extended using the polynucleotide as a template to produce an extension product, the oligonucleotide primer in (iii) binds to the extension product and is extended, resulting in cleavage of the extension product.

### BRIEF DESCRIPTION OF THE DRAWINGS

FIG. 1 illustrates probe-primer embodiments in which the probe segments are molecular beacons. In this illustration, R and Q are conjugated to 5′ and 3′ termini, respectively, of a stem-loop structure.

FIG. 2 illustrates probe-primer embodiments in which the probe segments are hydrolysis probes.

FIG. 3 illustrates the PCR amplification of a target sequence present in a double-stranded target polynucleotide. Two or more rounds of PCR amplification are carried out.

FIG. 4 illustrates an encoding amplification using the ligase chain reaction. Two or more rounds of PCR amplification are carried out.

FIG. 5A-5E illustrates an embodiment of a decoding step in which an amplicon, and by association a target sequence, is detected using PCR amplification.

FIG. 6A-6D illustrates an embodiment of a decoding step in which an amplicon, and by association a target sequence, is detected using PCR amplification and hydrolysis of the probe segment as the signal-generating mechanism.

FIG. 7 illustrates an embodiment of an encoding step, in which PCR amplification is used to associate two target sequences present in double-stranded target polynucleotides with different indexing sequences.

FIG. 8A-8D illustrates an embodiment of a decoding step in which two amplicons, and by association two target sequences, are detected and distinguished using PCR amplification and hydrolysis of probe segments as the signal-generating mechanism.

FIGS. 9-12 show exemplary methods for synthesis of exemplary multifunctional probes and component parts thereof.

### DETAILED DESCRIPTION

### 1. Definitions and Terminology

As used herein, a “sequence” means a nucleic acid base sequence of a polynucleotide. Unless otherwise indicated or apparent from context, bases or sequence elements are presented in the order 5′ to 3′ as they appear in a polynucleotide.

A “polynucleotide” refers to DNA (including, e.g., genomic, mitochondrial, viral, synthetic and cDNA), RNA (including, e.g., mRNA, siRNA, microRNA). Polynucleotides may include chimeric molecules, nucleic acids comprising non-standard bases (e.g., inosine), and other functional molecules. Polynucleotides may be single-stranded or double-stranded, Polynucleotides are at least 10 bases or basepairs in length. A polynucleotide having a length in the range of 10-70 bases, inclusive, can also be called an “oligonucleotide.”

A “target polynucleotide” is a polynucleotide that comprises a target sequence. In a double-stranded target polynucleotide the target sequence is on one strand and the complement of the target sequence is on the other strand.

The term “segment,” refers to a sequence or subsequence in a polynucleotide, such as a segment having a particular function, e.g., probe segment, binding segment, probe-binding segment, amplicon-binding segment, indexing sequence, and others listed herein. Individual segments may have any length consistent with their intended function, such as, without limitation, lengths in the range of 10-100 nucleotides, 10-70 nucleotides, 14-50 nucleotides, and 14-35 nucleotides.

A “target sequence” or “target segment” is a nucleic acid sequence detected in an assay. In most cases a target sequence of interest is predefined (i.e., sequence is known prior to analysis). In other cases the complete target sequence is not known, but is defined as the sequence that is amplified by primers of known sequence. A target sequence may be found in DNA (including genomic, mitochondrial, viral, synthetic and cDNA), in RNA, or in amplifiable synthetic analogs thereof.

Two target sequences are “distinct” if they differ from each other by at least one nucleotide.

As used herein, “complementary” has its normal meaning in the molecular biology arts, and refers to a relationship between two antiparallel nucleic acid sequences in which the sequences are related by the base-pairing rules: A pairs with T or U and C pairs with G. A first sequence or segment that is “perfectly complementary” to a second sequence or segment is complementary across its entire length and has no mismatches. A first sequence or segment is “substantially complementary” to a second sequence of segment when a polynucleotide consisting of the first sequence is sufficiently complementary to specifically hybridize to a polynucleotide consisting of the second sequence. For illustration, hybridization conditions are salt concentrations less than about 1.0 M sodium ion, typically about 0.01 to 1.0 M sodium ion at pH 7.0 to 8.3, and temperatures at least about 30° C. for polynucleotides 10 to 50 nucleotides in length and at least about 60° C. for longer probes (e.g., greater than 50 nucleotides). Typically, specific hybridization will occur when there is at least about 55% base complementary over a stretch of at least 14-25 nucleotides, preferably at least 65%, more preferably at least 75%, more preferably at least 85%, more preferably at least 90%, and most preferably at least 95%. The prime symbol [′] is used to indicate a perfectly or substantially complementary sequence.

The terms “anneal”, “hybridize” or “bind,” in reference to two polynucleotide sequences, segments or strands, are used interchangeably and have the usual meaning in the art. Two complementary sequences (e.g., DNA and/or RNA) anneal or hybridize by forming hydrogen bonds with complementary bases to produce a double-stranded polynucleotide or a double-stranded region of a polynucleotide.

Two sequences or segments in a polynucleotide are “adjacent” or “contiguous” if there is no intervening sequence or non-nucleotide linker separating them. In some contexts, “non-adjacent” refers to two probe-binding sequences separated from each other by an intervening target sequence.

Two sequences or segments in a polynucleotide are “close” when they are separated by fewer than 20 bases, often fewer than 10 bases, and sometimes within 1, 2, 3, 5, less than 10, or 10 bases. For example, probe-binding segments and indexing segments may be separated to facilitate binding of indexing primer.

The terms “amplicon” and “amplification product” are used interchangeably and have their usual meaning in the art. The grammatically singular term, “amplicon,” can refer to many identical copies of an amplification product. Moreover, reference to an “amplicon” encompasses both a molecule produced in an amplification step and identical molecules produced in subsequent amplification steps (such as, but not limited to, amplification products produced in subsequent rounds of a PCR amplification). Moreover, the term “amplification may refer to cycles of denaturation, annealing and extension, and does not require geometric or exponential increase of a sequence.

Two sequences are “associated” when the first and second sequences appear in the same single or double-stranded polynucleotide molecule or when the first sequence and the complement of the second sequence appear in the same polynucleotide strand. Two associated sequences may or may not be contiguous with each other.

A “primer” is an oligonucleotide or polynucleotide comprising a sequence that is complementary to, and capable of hybridizing to, a target sequence or an indexing sequence, or the complement thereof. In some cases a primer is extended by a DNA-dependent DNA polymerase. However, as the term is used herein, a “primer” may refer to an oligonucleotide that is not extended, such as “primers” that hybridize to a target sequence in ligase based amplification reactions. In general, “primer” means an “extendible primer” that can prime template-dependent DNA synthesis.

A “decoding primer” is a primer extension primer that specifically binds to an indexing sequence or complement thereof, or target-binding sequence or complement thereof, or target sequence or complement thereof, in an amplicon produced according to the process described herein, and which may be extended by a DNA polymerase.

A “signal moiety” is a molecule that contributes to generation of a detectable signal under certain conditions. A “Donor-Acceptor Pair” comprising a donor (reporter) and acceptor (quencher) fluorophores is an example of a signal moiety. As known in the art and described below, changing the spatial relationship of the reporter and quencher can generate a detectable signal. Signal moieties may include radioisotopes, fluorophores, chromophores (e.g., colored particles), mass labels, electron dense particles, magnetic particles, spin labels, and molecules that emit chemiluminescence. Methods for use of such labels are well known.

The terms “multiplex” and “multiplexing” refer to assays in which two or more primer sets are used to amplify two or more distinct target sequences in the same amplification reaction mixture.

A “amplification reaction mixture” is the solution in which an amplification reaction takes place and may comprise one or more of target polynucleotides, primers, polymerase, ligase, amplification reagents, amplicons, buffering agents, nuclease inhibitors, divalent cations, dNTPs, and/or other components known in the art for amplification.

As used herein, a “sample” refers to a composition containing a target polynucleotide. Exemplary samples include purified or partially purified DNA or RNA, cells and cell lysates (e.g., eukaryotic cells, human cells, animal cells, plant cells, stem cells, blood cells, lymphocytes, bacterial cells, recombinant cells and cells infected with a pathogen. tissue samples), viruses, environmental samples (e.g., water samples), food samples, forensic samples, plant samples, blood samples and the like. “Cell lysates” includes partially purified cell fractions.

As used herein, “amplification” of a nucleic acid sequence has its usual meaning, and refers to in vitro techniques for enzymatically increasing the number of copies of a target sequence. Amplification methods include both asymmetric methods (in which the predominant product is single-stranded) and conventional methods (in which the predominant product is double-stranded).

As used herein, “stem-loop” has its normal meaning in the art and refers to a polynucleotide secondary structure that arises when two regions of the same polynucleotide are inverted complements that anneal to form a double helix (“stem”) that ends in an unpaired loop.

Abbreviations: P—probe-binding sequence, I—indexing sequence; T—target-binding sequence; X—primer-binding sequence; π—amplicon-binding sequence; R—reporter; Q—quencher; L—linker.

Symbols: The “prime symbol” (′) indicates that a sequence (e.g., X′) is complementary to a corresponding sequence (e.g., X). The colon symbol (:) indicates that a first sequence segment (e.g., X′) is hybridized to a second sequence (e.g., X or π),e.g., “X′:X” or “X′:π”. An arrowhead on an oligonucleotide in a diagram indicates the 3′ end of the oligonucleotide.

### 2. Overview

In one aspect, the invention provides methods for detecting target nucleic acid sequences in a sample. A target sequence may be of interest because it is from a pathogenic organism, because it contains genetic information such as a single nucleotide polymorphism, deletion or insertion, because copy number variation of the sequence is informative of a subject's medical status, and for many other reasons that will be known to physicians and scientists. A target sequence may be found in a naturally occurring polynucleotide and/or may be from a non-naturally occurring polynucleotide such as an amplicon. For example, a sequence may be amplified from a naturally occurring template, optionally with addition of synthetic sequences, and the resulting amplicon sequence detected.

In some embodiments detection of target sequences in a sample can be described as a three-step process involving (1) an encoding amplification in which the target sequence is associated with probe-binding sequences and optionally with indexing sequences, (2) a distribution step in which the product of the encoding amplification is split into multiple aliquots, and (3) a decoding and detection step in which the presence, absence, quantity, or relative amount of the target sequence in the aliquots is determined. The determination of the presence, absence, quantity, or relative amount of the target sequence is indicative of the presence, absence, quantity, or relative amount of the target sequence in the initial sample. In some embodiments the detection of target sequences in a sample comprises (1) an encoding amplification and (2) a decoding and detection step, in which a distribution step is optional.

### 3. Multifunctional Probe-Primer Oligonucleotides

This invention utilizes composite oligonucleotides (“probe-primers”) that function as both probe and primer in assays for detecting target sequences. As used herein the “probe” (or “detection probe”) portion of the probe-primer comprises signaling moieties (e.g., reporter and quencher) and an amplicon binding sequence (π) that binds to probe-binding sequences in the amplicon (discussed below). The primer portion comprises an extendible 3′ terminus. The probe and primer portions are connected by a linker. One aspect of these probe-primers is that the probe segment or segments and the primer segment or segments are attached to each other at their 5′ ends. In some embodiments, the 5′ terminal nucleotide of one (or both) of the oligonucleotides connected to another oligonucleotide is covalently bound to the linker moiety. That is, a linker, such as a nonnucleotide linker, connects the primer segment and probe segment(s) in a 5′-5′ orientation. In an embodiment, the linker is covalently attached to the 5′ nucleotide of each probe and primer segment. However, linkages may also be with internal nucleotides close to the 5′ terminus (e.g., typically within 1, 2, 3, 5, less than 10, or bases of the 5′ terminus, but always closer to the 5′ terminus than to the 3′ terminus).

A probe segment of the probe-primers of this invention comprises an amplicon-binding sequence (π) that will hybridize to an amplicon generated in the encoding or decoding step (described below). A primer segment of the probe-primers of this invention also comprises an indexing primer sequence (I). Sequence I, or its complement, I′, is associated with the target sequence as part of the encoding step (described below).

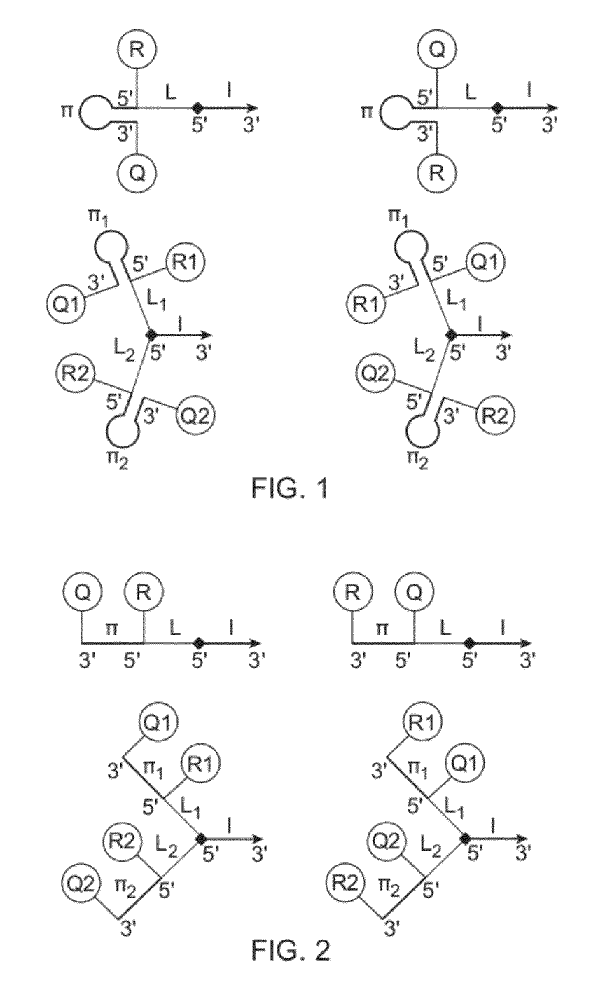

FIG. 1 shows diagrams of probe-primer embodiments where the probe segments are molecular beacons. The molecular beacons are drawn to show a polynucleotide with a stem-loop configuration, with a quencher (Q) conjugated to the 3′ terminus and a reporter (R) conjugated to the 5′ terminus. In certain embodiments the positions of Q and R may be reversed (e.g., Q at or near the 5′ terminus, and R at or near the 3′ terminus). In this context, “near” the terminus typically means within 1, 2, 3, 5, less than 10, or 10 bases of the 5′ or 3′ terminus. FIG. 2 shows diagrams of probe-primer embodiments where the probe segments are hydrolysis probes. In FIGS. 1 and 2, both single probe and multiple probe (“multiprobe”) constructs are shown.

### 4. Assays for Detecting Target Sequences

Assays that utilize the probe-primers of this invention comprise an encoding step and a decoding step. These two steps may be part of a single reaction (e.g., occur in a single reaction volume) or may be separated into more than one (e.g., two) reaction volumes.

4.1 Encoding Step

In the encoding step, a target sequence (or more typically, multiple different target sequences in a multiplex reaction) is associated with specified nucleic acid sequences referred to as “indexing sequences.” Optionally, the target sequence is also associated with one or more “probe-binding sequences.” This association is effected when a polynucleotide comprising the target sequence is reacted with an “encoding primer set.” For example and not for limitation, an encoding primer set may be a PCR primer pair, an LCR primer set, or other primers that may be used in amplification methods. Each encoding primer in an encoding primer set has a “target-binding” sequence or segment (T), and an “indexing” sequence or segment (I). Optionally, one or more encoding primers in an encoding primer set include a “probe-binding” sequence or segment (P). Typically, target-binding segments and indexing segments are 15-40 nucleotides in length and probe-binding segments are 5-20 nucleotides in length. These ranges are guidelines but are not intended to limit the invention. Although typically an encoding primer has two (T+I) or three (T+I+P) sequence elements, the presence of additional sequence elements is not excluded.

Various amplification and reaction methods may be used to associate indexing sequences, and optionally probe-binding sequences, with target sequences in an encoding step. For illustration, polymerase chain reaction (PCR) approaches in which the encoding primer set consists of two primers designed to amplify a specified target sequence under PCR amplification conditions, and polymerase chain reaction (LCR) approaches are discussed below.

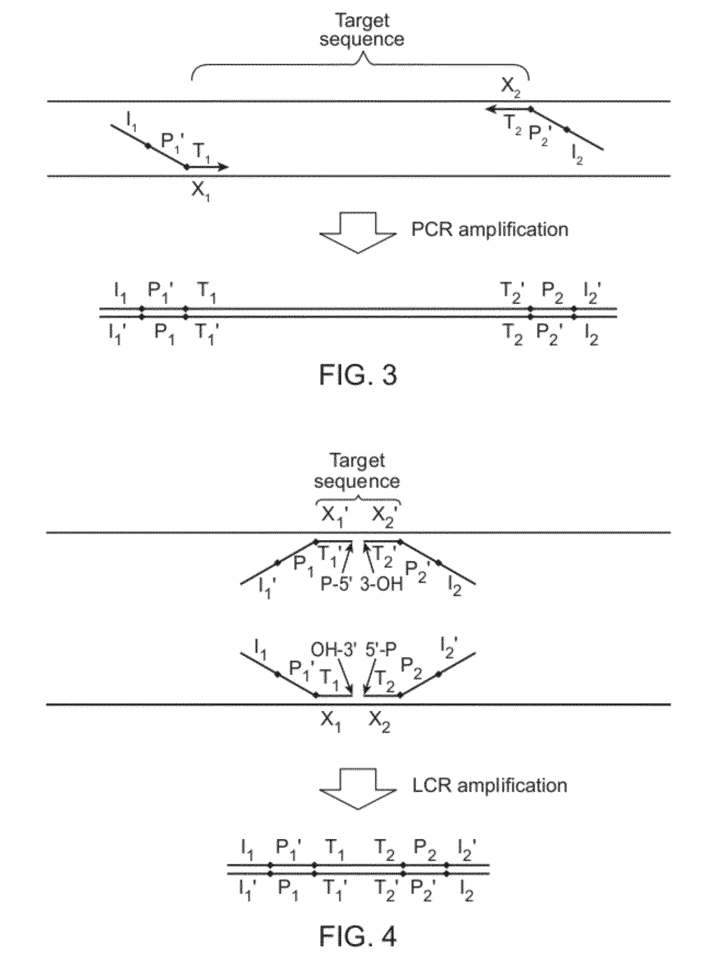

FIG. 3 illustrates the PCR amplification of a target sequence present in a double-stranded target polynucleotide. In PCR amplification approaches, the target-binding sequence T is located at the 3′ end of each encoding primer. The target sequence is in the upper strand with the boundaries shown, while the lower stand comprises the complement of the target sequence. As shown in FIG. 3, amplification produces an amplicon in which the target sequence is flanked by a first indexing sequence (I₁) and the complement of a second Indexing sequence (I₂′), a first target-binding sequence (T₁) the complement of a second target-binding sequence (T₂′), and optionally by one or more probe binding sequences and complement of probe binding sequences (P and P′).

In referring to the target-binding, probe-binding or indexing sequences, we will generally refer to a “pair of indexing sequences,” for example, rather than to the more cumbersome “first indexing sequence and complement of second indexing sequence.” It will be apparent to the reader, particularly with reference to the figures, when reference to a complementary sequence is intended or encompassed. The portion of the target polynucleotide to which the target-binding sequence (T) hybridizes is referred to as a primer-binding sequence (X). The target-binding sequence T of the encoding primer is sufficiently complementary to X to specifically hybridize to the target polynucleotide. If both target-binding sequences (T) are exactly complementary to the primer binding sequences (X) to which they bind, the sequence [T₁-target sequence-T₂′] in the amplicon will correspond exactly to a sequence in the polynucleotide template. When a target-binding sequence T is not exactly complementary to the primer binding sequence X, it will be at least sufficiently complementary to specifically hybridize. In addition, the sequence of a target binding sequence (T) may be further constrained by the requirements of the amplification method. For example, if the encoding primer is a PCR primer, the 3′ base should bind the corresponding base of the target sequence to promote primer extension.

Although for simplicity FIG. 3 shows amplification of a single target sequence, the encoding amplification is typically a multiplex reaction. That is, two or more different target sequences are amplified using different encoding primer sets. Usually at least two of the different target sequences become associated through the encoding amplification with different probe-binding sequences and/or different indexing sequences, such that the amplicons containing different target sequences can be distinguished from each other in the decoding step.

As noted above, amplification methods other than PCR methods may be used in the encoding step. For example, amplification may be carried out using the ligase chain reaction (LCR), in which case an encoding primer set may consist of four primers. FIG. 4 illustrates an encoding amplification using the ligase chain reaction (LCR, Barany 1991 Proc. Natl. Acad. Sci. 88:189-193, Barany 1991 Genome Res. 1:5-16, incorporated by reference herein). In this approach, the “encoding primer set” contains two pairs of primers, rather than one pair used in PCR methods. An encoding primer set used for such amplification includes a first primer, with a 3′ terminal segment sufficiently complementary to the target sequence to anneal to the target polynucleotide, and a second primer, with a 5′ terminal segment sufficiently complementary to anneal to the target polynucleotide (e.g., 5′-I₁-P₁′-T₁-3′ and 5′-T₂-P₂-I₂′-3′). The second pair comprises sequences complementary to the sequences of the first pair.

In conventional LCR methods, the primers are designed to anneal at adjacent sites on the target strand so that the 3′ base of one primer can be ligated directly to the 5′ base of the second primer. Alternate methods are known, however, including “Gap-LCR” (in which DNA polymerase is used to fill a gap between two annealed probes), PCR-followed by LCR, inclusion of a flap endonuclease (FEN) as part of the LCR method, and the like, which may be adapted for the present invention. See, e.g., Abravaya et al., 1995, Nuc. Acids Res. 23:675-682 and references cited therein, all incorporated by reference herein. Also see, e.g., Wiedmann et al., 1994 Genome Res. 3:S51-S64; Gill and Ghaemi, 2008, “Nucleic acid isothermal amplification technologies: a review” Nucleosides, Nucleotides & Nucleic Acids, 27:224-43; and Bi et al, U.S. Pat. No. 6,511,810, all incorporated by reference herein.

Ligation using one pair of encoding primers (e.g., 5′-I₁-P₁′-T₁-3′ and 5′-T₂-P₂-I₂′-3′) may be used in the encoding step. A single round of ligation or multiple rounds of denaturation and ligation may be employed. The use of multiple rounds of denaturation/ligation results in linear amplification of ligated encoding primers.

It will be appreciated that PCR, LCR, other amplification methods and conditions, and ligation methods and conditions are well known and need not be described in detail here. In general terms, the reaction mixture (the solution in which the amplification or ligation reaction takes place) may comprise sample (containing target polynucleotides), primers, amplification or ligation reagents, buffering agents, nuclease inhibitors, Mg⁺⁺ or other divalent cations, dNTPs, and other components known in the art for amplification or ligation, and amplicons. Enzymes that may be present include DNA polymerase (e.g., Taq polymerase or another thermostable DNA polymerase), ligase (e.g., bacteriophage T4 ligase, E. coli ligase, Afu ligase, Taq ligase). In some embodiments, the DNA polymerase has a 5′→3′ nuclease activity. It is routine in the art to design primers and select appropriate reaction conditions (e.g., slope of temperature ramp, duration and temperatures for denaturation, annealing and extension in PCR).

In one embodiment, all of the amplicons produced in a multiplex amplification reaction share the same combination of probe-binding sequences (e.g., all P₁+P₂), reflecting the use of encoding primers designed with the same probe binding sequences. In other embodiments, each of the amplicons produced in a multiplex amplification reaction has a different (unique) combination of probe binding sequences. Encoding primer sets that are different or distinct may have members in common. For example, a first set may comprise forward primer A and reverse primer C, and a second set may comprise forward primer B and reverse primer C.

4.2. Distribution Step

If the encoding step and decoding step are separated into two reactions (i.e., two reaction volumes or aliquots), then a distribution step may included between the encoding step and the decoding step. In the distribution step, the encoding amplification reaction mixture is divided into two or more separate aliquots, each of which can be independently (but usually simultaneously) assayed for the presence or absence of a target sequence. The presence or absence of a target sequence in an aliquot is indicative of the presence, absence, quantity, or relative amount of the target sequence in an initial sample selected for analysis.

In some embodiments, decoding primers and decoding probe-primers (both discussed below) are added to individual aliquots. That is, in some embodiments the distribution of a reaction mixture into aliquots comprises creating individual aliquots of the reaction mixture and then adding reagents (e.g., decoding primers and decoding probe-primers) to each aliquot. In a different embodiment, distribution of a sample into aliquots comprises combining the reaction mixture with reagents (e.g., decoding primers and decoding probe-primers), and then distributing the mixture into individual aliquots of the mixture without subsequent addition of additional reagents.

Specific methods for distribution are not critical to the practice of the invention, although they may be of practical significance. In one embodiment, distribution is carried out by manual pipeting. For example, ten 1-microliter aliquots of a 10-microliter reaction mixture may be distributed to tubes by pipeting. More typically, robotic methods are used and preferably microfluidic methods are used for efficiency and economy. In some embodiments the reaction mixture is divided into a plurality of aliquots of substantially equal volume.

For illustration and not limitation, a number of microfluidic devices are known for distribution of a sample or reaction mixture followed by addition of reagents. Fluidigm Corp. (South San Francisco Calif.) provides a number of platforms for distribution and combinatorial addition of reagents, including commercially available Dynamic Array™ and Access Array™ systems, and as well as systems described in the literature (see, e.g., U.S. Pat. No. 7,604,965; Patent publications WO 2010/077618; US 2009/0317798; US 2008/0223721; US 2009/0257920; US 2009/0291435; US2011/0126910 and unpublished application PCT/US10/58459, all incorporated by reference herein). Other approaches include use of microfluidic cards. One useful approach makes involves distribution of the reaction mixture into microdropletes in which amplification reactions may be carried out (see, e.g., Patent Application Publication Nos. US 2009/0035838; US 2010/0022414; WO 01/89788; WO 2006/040551; WO 2006/040554; WO 2004/002627; WO 2008/063227; WO 2004/091763; WO 2005/021151; WO 2006/096571; WO 2007/089541; WO 2007/081385 and WO 2008/063227, all incorporated by reference herein. In one droplet-based approach the sample may be partitioned into a plurality of droplets and individual same droplets fused with droplets containing specified reagents.

In another approach, partitioning methods are used. In these methods the encoded sample is combined with detection reagents (including decoding primers and decoding probe-primers) prior to distribution to separate compartments or droplets in which the detection reactions are carried out (see, e.g., U.S. Pat. No. 7,604,965, incorporated by reference herein). For example, the mixture can be distributed to individual aliquots by partition of a channel containing the mixture, by distribution of the mixture into droplets, and the like. In some embodiments a commercially available Digital Array™ partitioning device (Fluidigm Corp., South San Francisco, Calif.) is used.

4.3. Decoding and Detection Step

In the decoding step, amplicons produced in the encoding step are detected. Detection of an amplicon involves combining the amplicon with at least one of the probe-primers described above under conditions in which at least one probe segment hybridizes to at least one probe-binding segment, and detecting the hybridization. Without intending to limit the invention, in some embodiments hybridization is detected using a Molecular Beacon. Without intending to limit the invention, in some embodiments, detecting hybridization entails (1) hydrolyzing a portion of the probe segment in the presence of a decoding primer, thereby separating a signal moiety from the probe segment, and (2) detecting the separating of the signal moiety.

4.3.1 Detection Systems

Intramolecular binding of the detection probe portion of the amplicon to the probe-binding sequence can be detected in a number of ways, some of which are discussed below. Often, detection uses a fluorescence-based system. Typically, a system is used in which the intensity of a fluorescent signal is dependent on the proximity of a reporter and quencher to each other. For example, in some embodiments a detection probe is a dual labeled FRET (fluorescence resonance energy transfer) type probe. A FRET-based probe comprises a donor (reporter) and acceptor (quencher) fluorophore. The donor and acceptor fluorophore are selected such that the emission spectrum of the donor overlaps the excitation spectrum of the acceptor. Thus, when the pair of fluorophores are within sufficiently close proximity to one another, energy transfer from the donor to the acceptor can occur and can be detected as reduced signal relative to a donor not in proximity to an acceptor. A pair of fluorophores having this relationship above can be referred to as a “Donor-Acceptor Pair.” For purposes of terminology, a “Donor-Acceptor Pair” can be considered an example of a signaling moiety. A first donor-acceptor pair and a second donor-acceptor pair are considered different or distinct if they produce different detectable signals. Typically this means the two donor-acceptor pairs have different donor (reporter) fluorophores and optionally have different acceptor (quencher) fluorophores.

Signaling moieties for use in the invention are not limited to fluorescence-based systems. Any probe for which hydrolysis of the probe results in a detectable separation of a signal moiety from the detection probe-amplicon complex may be used. For example, in one approach, release of the signal moiety may be detected electronically (e.g., as an electrode surface charge perturbation when a signal moiety is released from the detection probe, amplicon complex) or chemically (e.g., a change in pH in a solution as a signal moiety is released into solution). In one embodiment, a reporter fluorophore is present, but no acceptor fluorophore is used. Hydrolysis resulting in release of the soluble reporter fluorophores is determined by physically removing the soluble reporter from the reaction mixture (e.g., by filtration) and hydrolysis is detected as a reduction in signal.

For convenience, exemplary fluorescence-based detection systems are sometimes described as using “hydrolysis probe systems” or “hybridization probe systems.” When “hydrolysis probe systems” are used, a reporter and quencher associated with the detection probe are separated by a cleavage of the probe so that the reporter and quencher are associated with different molecules which can become physically separated, resulting in a change in signal. For example, extension of an appropriately positioned decoding primer annealed to the amplicon using a polymerase with a 5′ nuclease activity (e.g., Taq polymerase) results in hydrolysis of all or a portion of the detection probe and release of the reporter (or quencher). Upon release, the quencher and reporter are physically separated, resulting in a detectable change in the fluorescent signal.

When “hybridization probe systems” are used, the reporter and quencher remain associated with the same detection probe molecule, but changes in the conformation of the probe increase or decrease the proximity of the reporter and quencher from each other, resulting in a change in signal. In some embodiments, the detection probe is a molecular beacon-type hybridization probe. In a molecular beacon, reporter and quencher molecules are linked at (or near) the 5′ and 3′ ends of an oligonucleotide that contains short (˜5 b.p.) complementary sequences of bases at the 5′ and 3′ ends. The complementary bases hybridize to form a stem-loop structure which holds the reporter and quencher close together in space at the base of the stem. When the probe hybridizes to another polynucleotide (e.g., an amplicon) it assumes a linear conformation in which the reporter and quencher are separated in space and the extent of quenching is diminished. Thus, hybridization of a molecular beacon-type probe segment to a probe-binding segment results in an increase of fluorescent signal.

Fluorescence-based systems are well known. See, e.g., Livak et al., 1995 “Oligonucleotides with fluorescent dyes at opposite ends provide a quenched probe system useful for detecting PCR product and nucleic acid hybridization” PCR Methods Appl. 4:357-362; Tyagi and Kramer, 1996, “Molecular beacons: probes that fluoresce upon hybridization” Nat. Biotechnol. 14:303-308; Piatek et al., 1998, Nat. Biotechnol. 16:359-63; Tyagi et al., 1998, Nat. Biotechnol. 16:49-53; U.S. Pat. No. 5,723,591; and U.S. Pat. No. 6,150,097 incorporated herein by reference). Exemplary reporters and quenchers include those described in Anderson et al, U.S. Pat. No. 7,601,821, incorporated herein by reference. Labeling probes can also comprise sulfonate derivatives of fluorescenin dyes with SO3 instead of the carboxylate group, phosphoramidite forms of fluorescein, phosphoramidite forms of CY 5 (available for example from Amersham). Exemplary reporter include fluorophores, such as a xanthene dye (e.g., fluorescein or a rhodamine), a cyanine dye, a dansyl derivative, EDANS, coumarin, Lucifer yellow, BODIPY, Cy3, Cy5, Cy7, Texas red, erythrosine, naphthylamine, or Oregon green, including 5-carboxyfluorescein (5-FAM); 6-carboxyfluorescein (6-FAM); 2′,4′,1,4,-tetrachlorofluorescein (TET); 2′,4′,5′,7′,1,4-hexachlorofluorescein (HEX); eosin; calcium green; NED; tetramethyl-6-carboxyrhodamine (TAMRA); tetrapropano-6-carboxyrhodamine (ROX); 2′,7′ dimethoxy-4′,5-dichloro-6-carboxyrhodamine (JOE); and tetramethylrhodamine. Exemplary quenchers include tetramethylrhodamine (TAMRA), DABCYL (DABSYL, DABMI or methyl red) anthroquinone, nitrothiazole, nitroimidazole or malachite green. Quenchers are also available from various commercial sources, such as Black Hole Quenchers® from Biosearch Technologies and Iowa Black® or ZEN quenchers from Integrated DNA Technologies, Inc.

It will be appreciated that detection may involve multiple rounds of amplification (e.g., to increase sensitivity). For example, a detection method may be carried out using of any of claims 32-33, wherein multiple rounds of denaturation, annealing and extension are carried out at least 5, at least 10, at least 15, or at least 20 rounds of denaturation, annealing and extension.

4.3.2 Decoding Step when Probe-Binding Segment (P) is not Included in Encoding Step

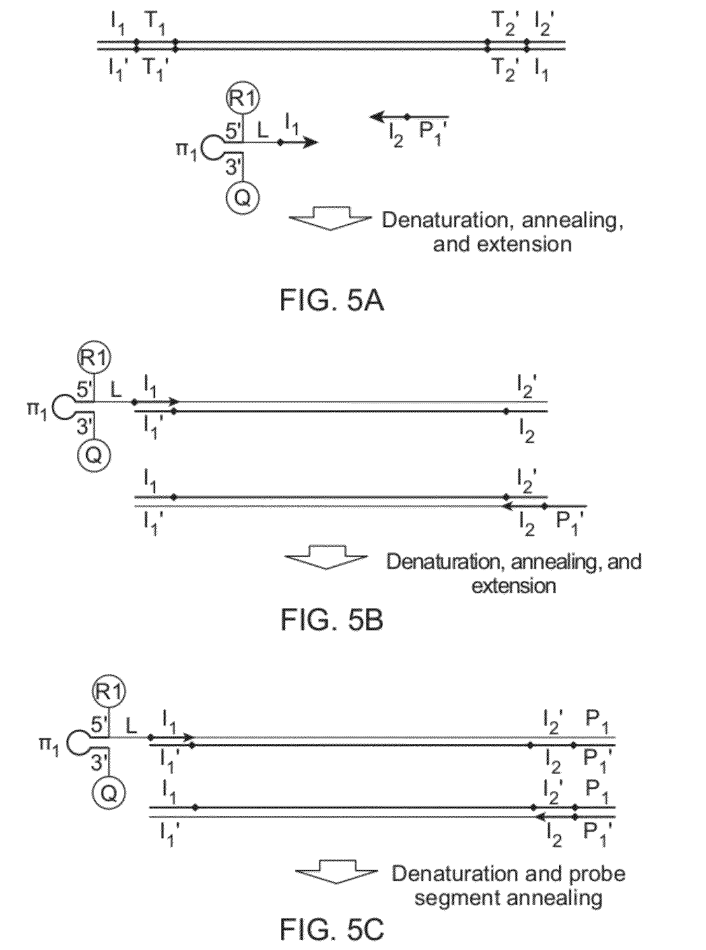

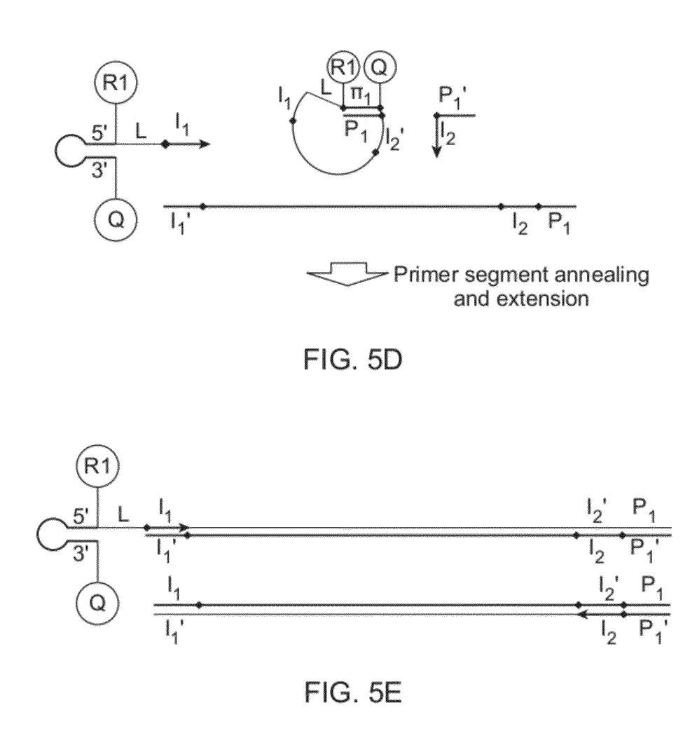

FIG. 5 illustrates one embodiment of the decoding step where an amplicon, and by association a target sequence, is detected using PCR amplification using a hybridization probe. In FIG. 5A, an amplicon generated in an encoding step is mixed with a probe-primer (comprising a probe segment with amplicon-binding sequence and a primer segment with an indexing sequence) and a decoding primer (comprising the complement of a probe-binding sequence and an indexing sequence, 5′-P₁′-I₂-3′). The amplicon is similar to that generated in FIG. 3, except that the amplification primers used did not include probe binding sequences. FIG. 5B shows the extension products generated after one round of denaturation, primer annealing, and extension with a DNA polymerase. FIG. 5C shows the extension products generated after the second round of denaturation, primer annealing, and extension with a DNA polymerase. In the next (3^{rd}) round and each subsequent round of amplification, a probe segment annealing temperature is included in the thermal protocol so that the amplicon-binding sequence (π₁) of the probe segment hybridizes to the probe-binding sequence (P₁) in the amplicon as shown in FIG. 5D. It will be recognized that the length and or other properties of the target sequence and linker are sufficient to allow intramolecular hybridization of π and P. As a result of the hybridization, the reporter (R) and quencher (Q) are separated in space. It is this hybridization event that generates the fluorescent signal that indicates that the amplicon is present, and by association, indicates that the target sequence is present in the original sample. One advantage of the probe-primers of this invention is that this hybridization occurs as a unimolecular or intramolecular reaction, which means that the hybridization occurs faster than it would if the hybridization event were a bimolecular reaction. The faster hybridization due to the unimolecular reaction means that the fluorescent signal generated during each round of amplification is typically stronger than in assays where the signal-generating hybridization event occurs as a bimolecular reaction. It will be appreciated that the target sequence is long enough to allow the unimolecular hybridization reaction to occur. FIG. 5D also shows the probe-primer, decoding primer and complementary strand of the amplicon present in the reaction mixture. After detection of fluorescent signal, the temperature is lowered to a primer segment annealing temperature and primer extension is initiated. FIG. 5E shows the resulting primer extension products, which are the same as in FIG. 5C. The process of denaturation, probe segment annealing, primer segment annealing, and extension is repeated as needed.

4.3.3 Decoding Step When Probe-Binding Segment is Included in Encoding Step

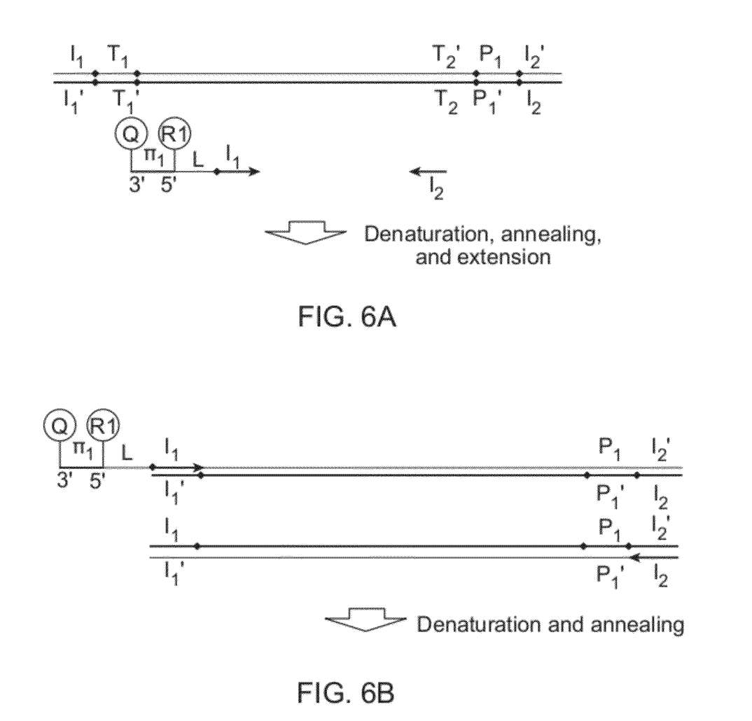

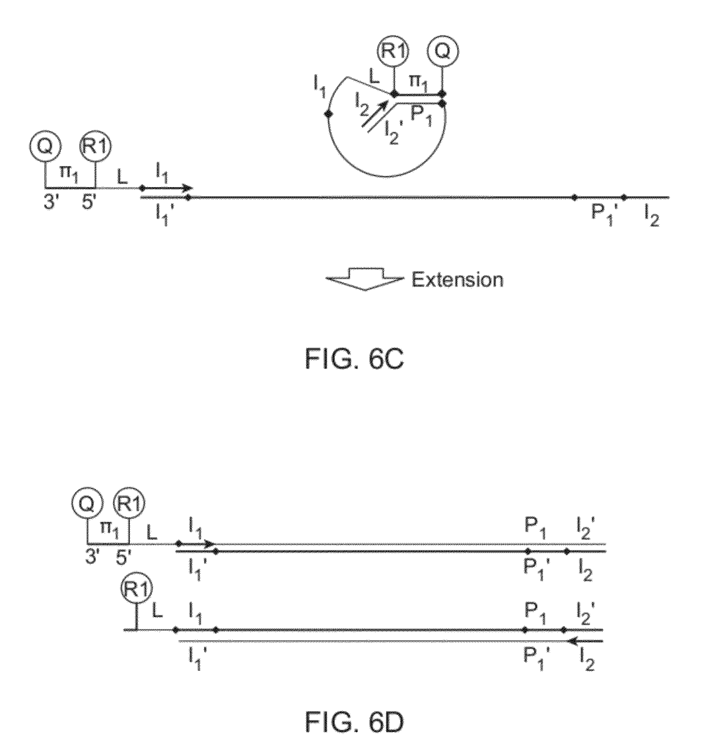

FIG. 6 illustrates one embodiment of the decoding step where an amplicon, and by association a target sequence, is detected using PCR amplification and hydrolysis of the probe segment as the signal-generating mechanism.

In FIG. 6A, an amplicon generated in an encoding step (see FIG. 3) is mixed with a probe-primer (comprising a probe segment with amplicon-binding sequence and a primer segment with an indexing sequence) and a decoding primer (comprising an indexing sequence, 5′-I₂-3′). FIG. 6B shows the extension products generated after one round of denaturation, primer annealing, and extension with a DNA polymerase. FIG. 6C shows the products generated after the second round of denaturation and annealing. In this case, there does not need to be a separate probe segment annealing temperature and primer segment annealing temperature. The unimolecular hybridization of the probe segment (P) with its complement (π) will occur faster than the bimolecular hybridization with its complement, so this embodiment still has the advantage of increased signal compared to assays where hybridization of the signal-generating probe is a bimolecular reaction. It will be appreciated that the target sequence is long enough to allow the unimolecular hybridization reaction to occur. FIG. 6D shows the extension products generated after primer extension of a primer comprising I₂ with a DNA polymerase that has 5′→3′ nuclease activity. Cleavage of the probe segment by the 5′→3′ nuclease activity physically separates the reporter and quencher, resulting in an increase in fluorescent signal. The process of denaturation, annealing, and extension is repeated as needed. In some embodiments, the primer-probe is designed so that extension (e.g., of the I₂ primer) cleaves (e.g., breaks or hydrolyses) a non-nucleotide bond through which a reporter or quencher is conjugated to the probe segment.

In the illustration shown in FIG. 6, the detectable probe segment is a FRET probe and the signal moiety, R1, is a reporter dye. In this FRET approach, partial hydrolysis of detectable probe results in physical separation of the reporter dye from a quencher dye, resulting in a detectable change in the fluorescent signal. However, the invention is not limited to FRET detection, and a variety of signal moieties and probe detection methods. The scheme shown in FIG. 6A-6E can also be carried out using a hybridization probe (e.g., Molecular Beacon).

### 5. Design of Primers, Primer Segments, and Detection Probe Segments

Guided by this specification, those of skill in the molecular biology arts will be able to design detection probe segments, primer segments, encoding primer sets, and decoding primers suitable for the practice of the invention. Parameters to be considered in probe and primer design include sequence length, secondary structure, dimer formation, GC content, reaction temperature conditions (e.g., denaturation, annealing and extension temperatures in the case of PCR), reaction salt and pH conditions, amplicon length and position, the melting temperature of the amplification product and the like. A variety of well-known methods and computer tools may be used to assist in design. See, e.g., Burpo, 2001, “A critical review of PCR primer design algorithms and cross-hybridization case study” Biochemistry 281:1-11; Rychlik et al., 1990, “Optimization of the annealing temperature for DNA amplification in vitro” Nuc. Acid. Res. 18:6409-11; SantaLucia, 1998, “A unified view of polymer, dumbbell, and oligonucleotide DNA nearest-neighbor thermodynamics” PNAS 95:41460-65; Lowe et al., 1990, “A computer program for selection of oligonucleotide primers for polymerase chain reactions” Nucleic Acids Res. 18:1757-61. Sambrook et al., MOLECULAR CLONING: A LABORATORY MANUAL (C.S.H.P. Press, NY 2d ed., 1989); and Ausubel et al., CURRENT PROTOCOLS IN MOLECULAR BIOLOGY, Greene Publishing and Wiley-Interscience, New York (1997), all incorporated by reference herein. In describing these methods general familiarity with amplification methods, including quantitative PCR methods has been assumed. For background, see, e.g., Bustin SA (ed), 2004, A-Z OF QUANTITATIVE PCR. La Jolla, Calif.: IUL Biotechnology Series, International University Line, incorporated by reference herein. As used herein “amplification conditions” refers to target and reagent concentrations, temperature (including thermocycling profiles), buffer compositions, and the like necessary to amplify a sequence using PCR, LCR or other amplification approaches. Amplification conditions are well known in the art, and are described in the scientific and patent literature, including but not limited to references cited herein.

In certain embodiments, for illustration, and without limitation, primer and probe segments described herein (e.g., P, I, T, X segments) may have lengths in the range of 10-30 bases. In certain embodiments the segments may have lengths in the range of 15-25 bases, or 10-50 bases.

### 6. Discrimination Assays for Distinguishing Target Sequences

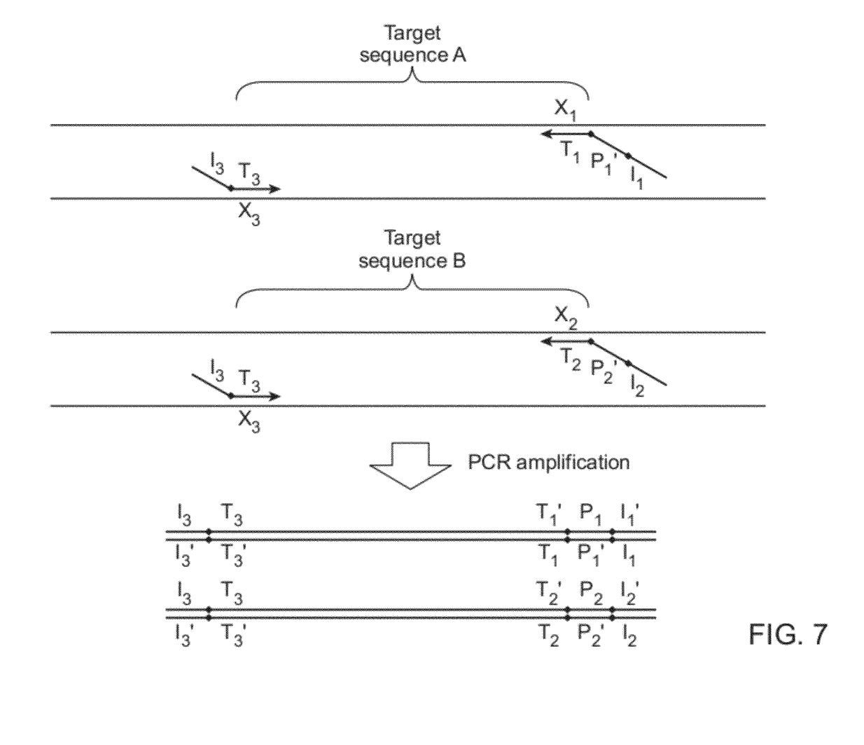

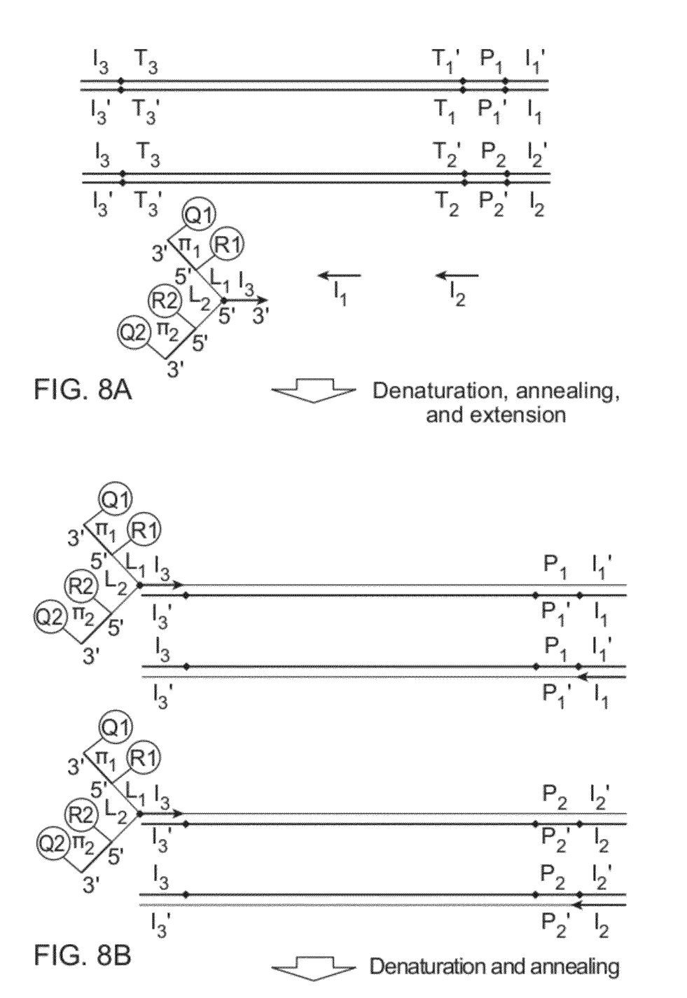

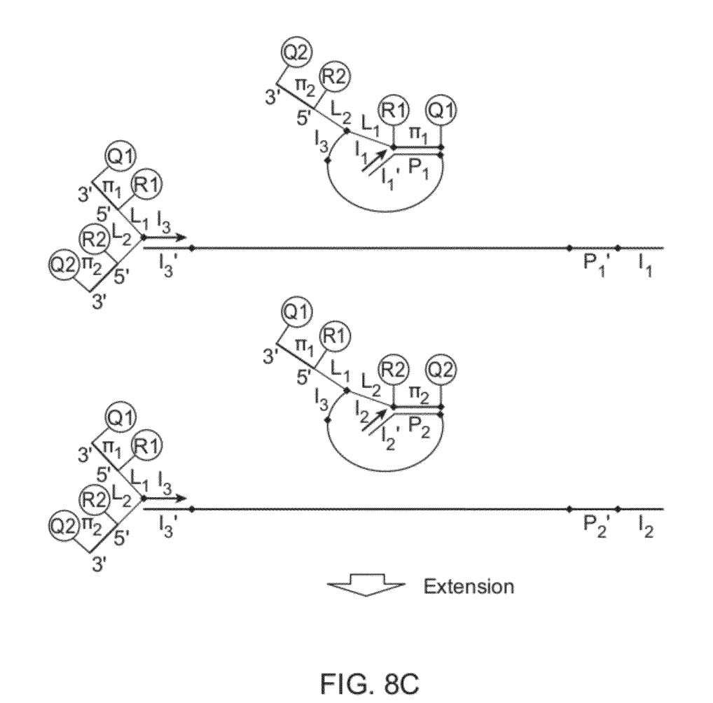

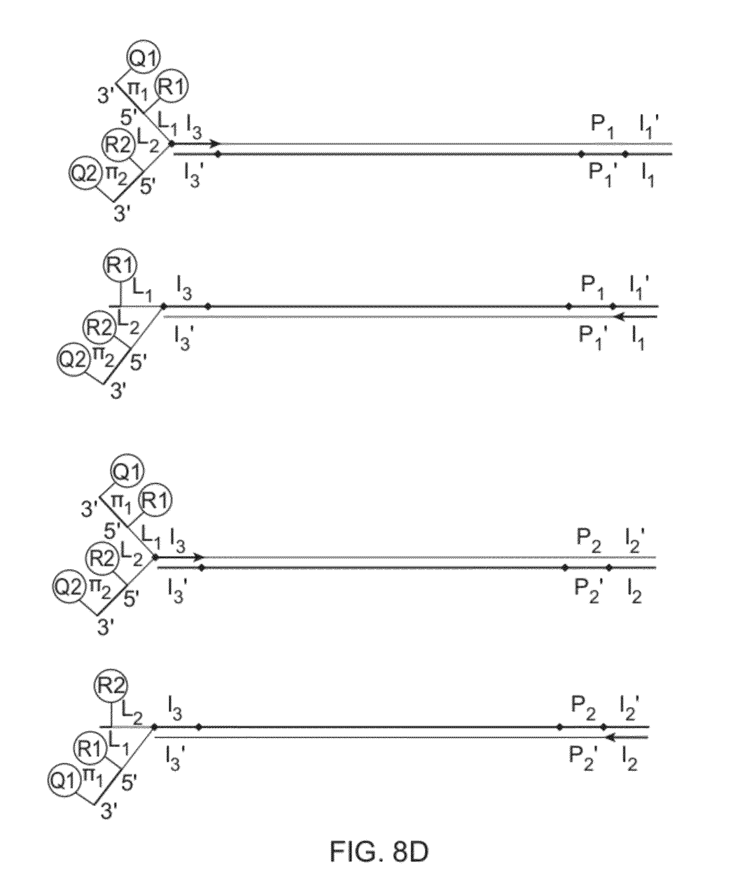

Assays that utilize the probe-primers of this invention can be used to distinguish two target sequences that differ by as little as a single nucleotide. These assays for discrimination of target sequences are comprised of encoding step, optional distribution step, and decoding step. The distribution step is the same as described in Section 5.2. We will first provide an overview of the encoding and decoding steps by referring to FIGS. 7 and 8, which illustrate an exemplary embodiment.

6.1 Encoding Step for Discrimination Assays

FIG. 7 illustrates one embodiment of the encoding step where PCR amplification is used to associate two target sequences present in a double-stranded target polynucleotides with different indexing sequences. These two target sequences may be present in the same sample (e.g., the same reaction volume) or in different samples.

Referring to FIG. 6, target sequence A will be distinguished from target sequence B based on sequence difference in primer-binding segments X₁ and X₂. If segments X₁ and X₂ differ by a single nucleotide, then, in a preferred embodiment, the single nucleotide difference is set to be the 5′ nucleotide of X₁ and X₂, respectively. This corresponds to a single nucleotide difference at the 3′ end of target-binding segment T₁ and target binding segment T₂, respectively. The encoding primer set consists of three primers comprising the following segments: 5′-I₁-P₁′-T₁-3′; 5′-I₂-P₂′-T₂-3′; and 5′-I₃-T₃-3′. As shown in FIG. 7, amplification using the encoding primer set produces two distinct amplicon in which the target sequence A is flanked by a first indexing sequence (I₃) on one side and a first probe-binding sequence (P₁) and the complement of a second Indexing sequence (I₁′), and target sequence B is flanked by a first indexing sequence (I₃) on one side and a second probe-binding sequence (P₂) and the complement of a third Indexing sequence (I₂′).

6.2 Decoding Step for Discrimination Assays

FIG. 8 illustrates one embodiment of the decoding step where two amplicons, and by association two target sequences, are detected and distinguished using PCR amplification and hydrolysis of probe segments as the signal-generating mechanism. It will be apparent to those of skill that this method may be extended for use with more than two target sequences.

FIG. 8A shows two amplicons generated in an encoding step (see FIG. 7). The amplicons comprise different target sequences (T₁ and T₂). Each amplicon comprises one indexing sequence in common (I₃), and unique indexing sequences (I₁, I₂) and probe binding sequences (P₁, P₂) that identify the target sequence. The amplicons are combined with a single probe-primer and two decoding primers (comprising indexing sequences I₁′ and I₂). The probe-primer comprises (a) two probe segments, each with amplicon-binding sequences (π) complementary to the target-specific probe binding sequences and a signal moiety specific (e.g., R₁, R₂) for each unique π sequence, and (b) a “decoding” primer sequence complementary to the common indexing sequence (I₃). FIG. 8B shows the extension products generated after one round of denaturation, primer annealing, and extension with a DNA polymerase. FIG. 8C shows the products generated after the second round of denaturation and annealing. Amplicon strands in which primer-probes have been incorporated are shown in a conformation in which π and P sequences are annealed. FIG. 8C also shows the decoding primers annealed to amplicon strands. FIG. 8D shows the extension products generated after primer extension with a DNA polymerase that has 5′→3′ nuclease activity, resulting in release of quenchers Q1 and Q2 and increase in signal from R₁ and R₂. In this manner, detection of the reporter R1 signal indicates the presence of target sequence A in the original sample, and detection of the reporter R2 signal indicates the presence of target sequence B In the original sample. In the illustration shown in FIG. 8, the detectable probe segments are FRET probes and the signal moieties, R1 and R2, are reporter dyes. In this FRET approach, partial hydrolysis of detectable probes results in physical separation of the reporter dye from a quencher dye, resulting in a detectable change in the fluorescent signal. However, the invention is not limited to FRET detection, and a variety of signal moieties and probe detection methods, some of which are discussed elsewhere herein, may be used. The process of denaturation, annealing, and extension is repeated as needed. It will also be recognized that, the multiplex detection of both target sequence A and target sequence B can be carried out in a single reaction volume, or in separate reaction volumes. Moreover, as is well known from basic encoding methods, the unique indexing sequences (I₁, I₂) and probe binding sequences (P₁, P₂) may identify that a class of target sequences rather than a single sequence. That is, P₁+I₁′ can identify a diverse set of target sequences provided the amplicons are all tagged with P₁′+I₁.

### 7. Universal and Indexing Sequences

Probe-binding (P) and π sequences can be designated as universal or indexed. Likewise, indexing sequences can be designated as universal or, more often, are indexed. The designations “universal” and “indexed” describe the relationship, in a specified assay, of probe-binding or indexing sequences and the target sequences with which they are associated. A probe-binding sequence or indexing sequence that does not uniquely identify the target sequence with which it is associated can be considered a “universal” sequence. For example, a sample may be assayed for the presence of 48 target sequences by associating each of the 48 target sequences with the same probe-binding sequence and detecting amplicons containing the probe-binding sequence using a single “universal” detection probe segment with the same π sequence that recognizes the probe-binding sequence. Each of the 48 target sequences could be associated with different indexing sequences, and distinguished from each other using 48 different decoding primers. Alternatively, a sample could be assayed for the presence of 48 target sequences by associating each of the 48 target sequences with a different probe-binding sequence. Each of the 48 target sequences could be distinguished based on annealing of one of 48 uniquely labeled detection probe-primers. In this case, each of the 48 target sequences could be associated with the same indexing sequence, and a single “universal” decoding primer could be used in detection of the 48 targets.

It will be apparent that, if desired, using indexed probe-binding sequences and/or indexing sequences can be used to selectively detect individual target sequences or defined subgenuses of target sequences. By using various combinations of indexing sequences and indexed probe-binding sequences analyses of considerable complexity can be carried out.

### 8. Linkers

A variety of linkers are known and may be used to produce multifunctional probe-primers of the invention. Typically the linker is a non-nucleotide linker. The chemical constituents of suitable linkers or spacers will be familiar to persons skilled in the art. However, the invention is not limited to any particular types of linkers.

Examples of linkers commonly used in combination with oligonucleotides includes oligo and polyethylene glycol (e.g., HEG, TEG, PEG), glycerol, 1′2′-dideoxyribose, and C2 alkyl-C15 alkyl spacers. See, e.g., WO 2007/114986, paragraphs [00181]-[00187] incorporated herein by reference, for descriptions of linkers. Branched linkers, such as dendrimers, may be used. Examples of suitable molecules include glycerol or substituted glycerol (e.g., 2-hydroxymethyl glycerol, levulinyl-glycerol); tetraminobenzene, heptaminobetacyclodextrin, 1,3,5-trihydroxycyclohexane, pentaerythritol and derivatives of pentaerythritol, tetraminopentaerythritol, 1,4,8,11-tetraazacyclo tetradecane (Cyclam), 1,4,7,10-tetraazacyclododecane (Cyclen), polyethyleneimine, 1,3-diamino-2-propanol and substituted derivatives, [propyloxymethyl]ethyl compounds (e.g., “trebler”), polyethylene glycol derivatives (e.g., Gnanou et al., 1988, Makromol. Chem. 189:2885; Rein et al., 1993, Acta Polymer 44:225, Merrill et al., U.S. Pat. No. 5,171,264, incorporated herein by reference for all purposes). Numerous dendrimers are known, e.g., Exemplary dendrimers for use in the present invention include, for illustration and not limitation, amine-terminated polyamidoamine (PAMAM), polyethyleneimine and polypropyleneimine dendrimers, and “dense star” polymers and “starburst” polymers such as those described in U.S. Pat. Nos. 4,587,329; 5,338,532; and 6,177,414, incorporated herein by reference for all purposes.

FIG. 1 illustrates a preferred configuation of three polynucleotides in a multiprobe construct, in which the two probe moieties each are directly linked to the primer (through two linker molecules or a single “branched” linker molecule; see below). This configuration can be referred to as “non-linear” or “branched”. In an alternative approach, a linear configuration can be used, in which a first probe moiety is linked to a second probe moiety and the second probe moiety is linked (in a 5′-5′ orientation) to the primer moiety.

In embodiments in which a first polynucleotide is linked to two other polynucleotides, e.g., multiprobes as shown in FIG. 1, the construct is sometimes described herein as comprising two linkers. It will be recognized, however, that single molecule linkers able to link three oligonucleotides are well known (e.g., “trifunctional linkers” or “branched linkers”) and may be used in any embodiments described herein. Thus, in certain embodiments of the invention comprising three linked polynucleotides, they are linked by a single linker molecule.

### 9. Kits

Materials and reagents for use in methods of the invention may be provided in kit form. For example and not limitation, kits can include one of more of the following reagents (i) a primer-probe described herein; (ii) one or more amplification primers (e.g., PCR or LCR primers) comprising a sequence of, or alternatively complementary to, the amplicon-binding sequence of a probe-primer, and optionally comprising a sequence of, or alternatively complementary to, an indexing primer; (iii) one or more indexing primers complementary to an indexing sequence of an encoding (amplification) primer. In one embodiment the kit comprises a set comprising a probe(s) and encoding (amplification) primer(s) that may be used in combination for the methods of the invention (including multiplex methods). In one embodiment the kit comprises a set comprising a probe(s), an encoding (amplification) primer(s) that may be used in combination for the methods of the invention, and an indexing primer(s) that may be used in combination for the methods of the invention (including multiplex methods). The reagents may be sold and/or packaged together and may be accompanied by instructions for use. The kit may also include other elements such as buffers, controls, and enzymes (such as a DNA polymers or DNA ligase).

### 10. Diagnostic and Other Applications

It will be apparent to the reader that the reagents and methods described here may be used for any number of assays, including assays in biomedicine, genetic research, agriculture, environmental and evolutionary studies, forensics, pharmacogenomic determinations, veterinary diagnostic testing, genomics and the like. In some aspects, the reagents and methods find use in diagnostic assays, including SNP analysis, detection of infectious agents (e.g., bacteria and virus), genetic screening, and single cell analysis.

### 11. Exemplary Methods for Producing Primer-Probes

11.1: One Primer Conjugated to One Molecular Beacon-Type Probe Labeled with 3′-Dabsyl/5′-FAM

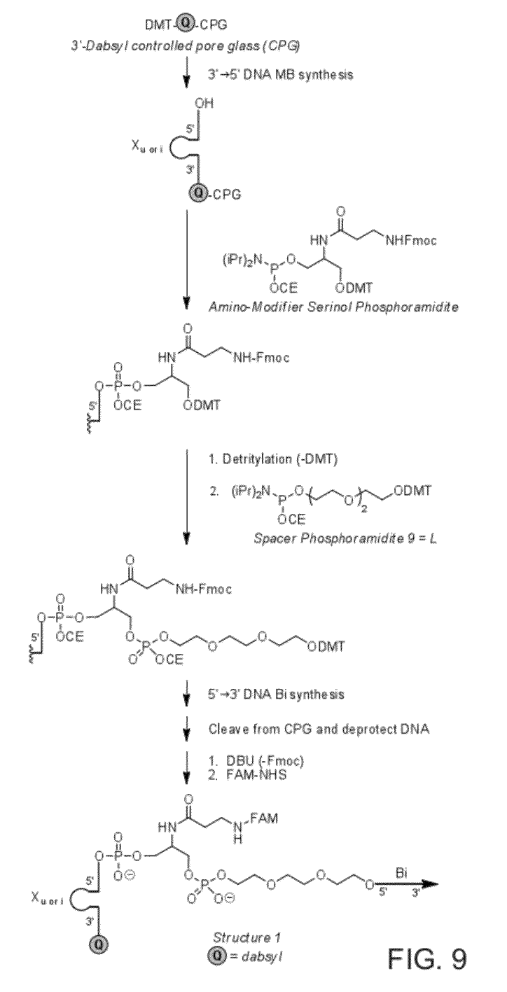

As shown in FIG. 9, commercially available (Glen Research Corp. Sterling Va.) 3′-Dabsyl CPG is used for conventional 3′→5′ solid-phase DNA synthesis of a desired Molecular Beacon sequence. The resultant 3′-Q-CPG/5′-OH intermediate is coupled with commercially available (Glen) Amino-Modifier Serinol Phosphoramidite according to vendor-recommended procedures to afford an orthogonally-protected NH-Fmoc/O-DMT intermediate. Detritylation (-DMT) of this intermediate is followed by coupling commercially available (Glen) Spacer Phosphoramidite 9 according to vendor-recommended procedures to install a poly(ethylene glycol) (PEG) linker (L). Detritylation of this PEG-linker moiety is followed by conventional “reverse” 5′→3′ DNA synthesis of Bᵢ using commercially available (Glen) 5′-phosphoramidte reagents according to vendor-recommended procedures. Cleavage from CPG and deprotection of DNA using conventional methods is followed by reaction with diaza(1,3)bicyclo[5.4.0] undecane (DBU) to remove the Fmoc (-Fmoc) moiety for conjugation of the resultant amino group with commercially available (AnaSpec) FAM-NHS ester according to vendor-recommended procedures to afford final product Structure 1.

It should be understood that the aforementioned synthetic route is applicable to other Q/R pairs using commercially available reagents and vendor-recommended procedures. For example, Q=Epoch Eclipse™ Quencher (Glen) or Black Hole Quencher® (Biosearch); and R=ROX, TAMRA, and TET (all from AnaSpec) or Cy3 and Cy5 (both from GE Healthcare).

It should be understood further that the aforementioned synthetic route is applicable to other Linker (L) moieties using commercially available reagents and vendor-recommended procedures. For example, Spacer Phosphoramidite 18 (PEG; Glen) or Spacer Phosphoramidites C3 and C12 (both CH₂-type and from Glen).

11.2: One Primer Conjugated to One Molecular Beacon-Type Probe Labeled with 3′-FAM/5′-Dabsyl

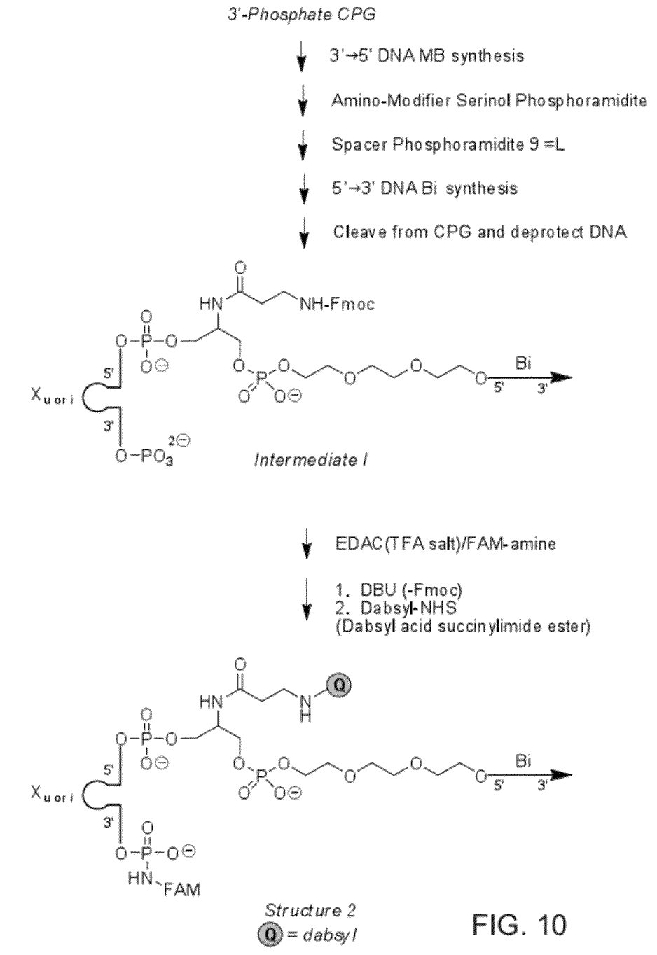

As shown in FIG. 10, commercially available (Glen) 3′-Phosphate CPG is used for the same series of synthetic steps as described in Example 1 through cleave from CPG and deprotect DNA, all according to vendor-recommended procedures. Resultant 3′-PO₃⁻²/5′NH-Fmoc Intermediate I is then reacted with commercially available (Pierce) 1-ethyl-3-[3-dimethylaminopropyl]carbodiimide hydrochloride (EDAC) and FAM-amine (AnaSpec HiLyte 488 amine, TFA salt) according to vendor-recommended procedures. This FAM-labeling step is followed by reaction with diaza(1,3)bicyclo[5.4.0]undecane (DBU) to remove the Fmoc (-Fmoc) moiety for conjugation of the resultant amino group with commercially available (AnaSpec) Dabsyl NHS-ester according to vendor-recommended procedures to afford final product Structure 2.

It should be understood that the aforementioned synthetic route is applicable to other Q/R pairs using commercially available reagents and vendor-recommended procedures. For example, Q=Epoch Eclipse™ Quencher (Glen) or Black Hole Quencher® (Biosearch); and R=ROX, TAMRA, and TET (all from AnaSpec) or Cy3 and Cy5 (both from GE Healthcare).

It should be understood further that the aforementioned synthetic route is applicable to other Linker (L) moieties using commercially available reagents and vendor-recommended procedures. For example, Spacer Phosphoramidite 18 (PEG; Glen) or Spacer Phosphoramidites C3 and C12 (both CH₂-type and from Glen).

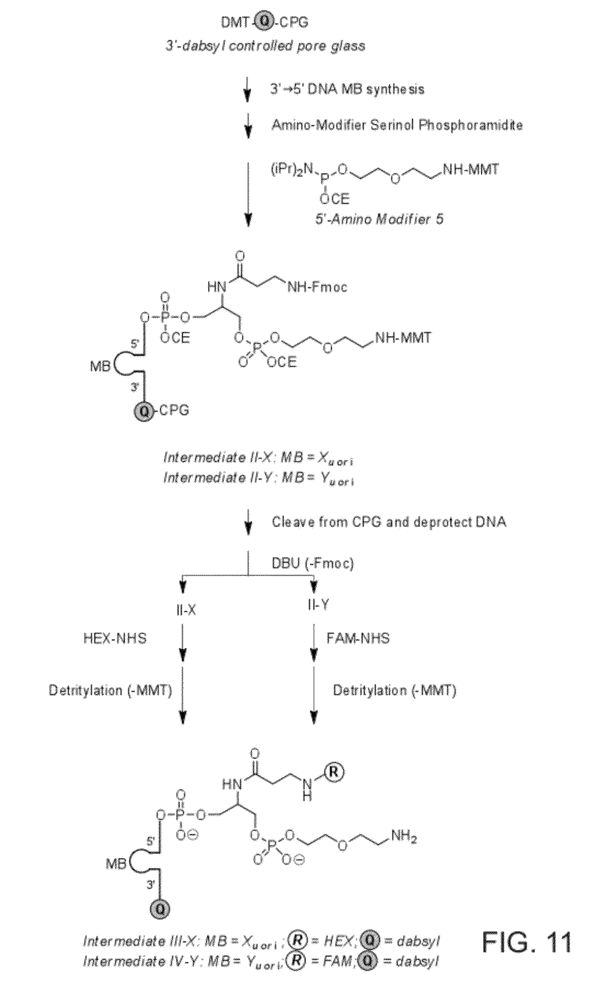

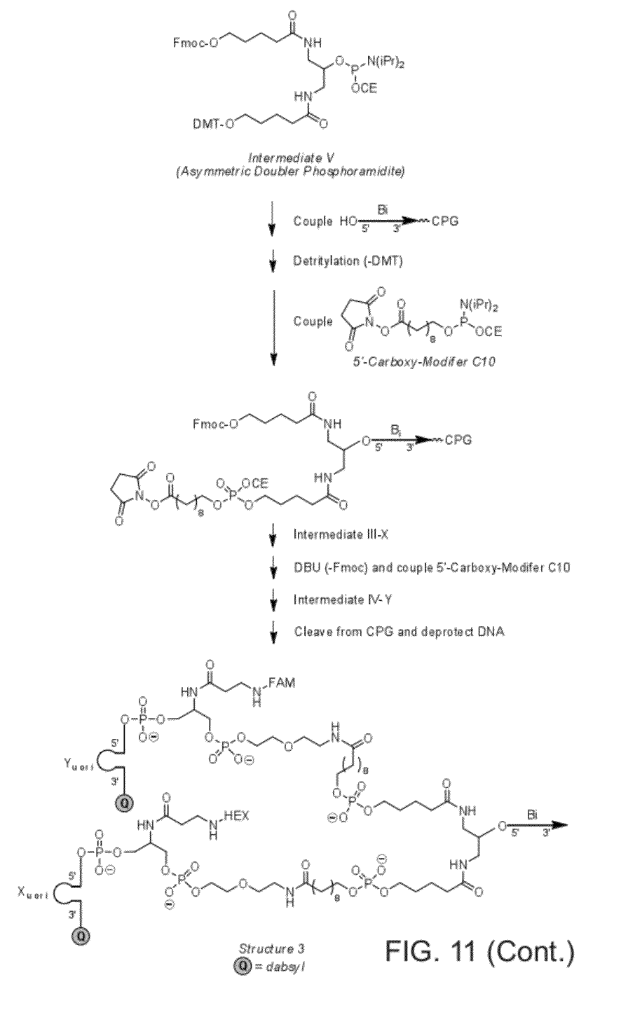

As shown in FIG. 11, one object of this invention utilizes a configuration wherein a 15-40 nt indexed Primer Binding Sequence (here designated Bᵢ) is conjugated to two different Molecular Beacon (MB) probes via a non-nucleic acid Linker (L). The first MB probe in this configuration is comprised of a 5-20 nt amplicon-binding sequence (here designated X_{u or i}) loop-region flanked by ˜5 nt self-complementary stem-sequences having attached Quencher (Q) and Reporter (R1) moieties that can be either 3′-Q/5′-R1 or 3′-R1/5′Q, as has been discussed elsewhere herein. The second MB portion of this configuration is comprised of a 5-32 nt amplicon-binding sequence (here designated Y_{u or i}) loop-region flanked by ˜5 nt self-complementary stem-sequences having attached Quencher (Q) and Reporter (R2) moieties that can be either 3′-Q15′-R2 or 3′-R2/5′Q, as has been already discussed in this provisional application. R1 and R2 thus differentiate target-specific detection by X_{u or I} and Y_{u or i}, respectively. Example 3 below specifies a synthetic scheme to obtain an embodiment with 3′-Q/5′-R1 and 3′-Q/5′R2, wherein Q=4-N,N-dimethylaminoazobenzene-4′-sulfonyl (Dabsyl), R1=fluorescein (FAM), and R2=6-carboxy-2′,4,4′,5′,7,7′-hexachlorofluorescein (HEX). Example 4 below specifies a synthetic scheme to obtain the “switched” embodiment with 5′-Q/3′-R1 and 5′-Q/3′R2, wherein Q=4-N,N-dimethylaminoazobenzene-4′-sulfonyl (Dabsyl), R1=fluorescein (FAM), and R2=6-carboxy-2′,4,4′,5′,7,7′-hexachlorofluorescein (HEX). In Sections 9.3 and 9.4, Bᵢ and X_{u or i}/Y_{u or i} sequences are depicted in generalized forms designated by a 5′→3′ arrow for Bᵢ and a stem-loop structure for X_{u or i}/Y_{u or i.}

11.3: One Primer Conjugated to Two Different Molecular Beacon-Type Probes Labeled with 3′-Dabsyl/5′-FAM and 3′-Dabsyl/5′-HEX

As shown in FIG. 10, commercially available (Glen) 3′-Dabsyl CPG is used for conventional 3′→5′ solid-phase DNA synthesis of a desired first-MB sequence (X_{u or i}) followed by sequential coupling with commercially available (Glen) Amino-Modifier Serinol Phosphoramidite and then 5′-Amino-Modifier 5, both according to vendor-recommended procedures, to afford orthogonally-protected NH-Fmoc/NH-MMT Intermediate II-X. These synthetic steps are repeated for a desired second-MB sequence (Y_{u or i}) to afford orthogonally-protected NH-Fmoc/NH-MMT Intermediate II-Y. Separate cleavage from CPG and deprotection of DNA for Intermediate II-X and Intermediate II-Y and then reaction with diaza(1,3)bicyclo[5.4.0]undecane (DBU) to remove the Fmoc (-Fmoc) moiety for conjugation of the resultant amino group with commercially available (AnaSpec) HEX-NHS ester and FAM-NHS ester, respectively, according to vendor-recommended procedures. Separate detritylation (-MMT) thus affords two different amino intermediates for subsequent conjugation, namely, Intermediate III-X; R(2)=HEX; X_{u or i} and Intermediate III-Y; R(1)=FAM; Y_{u or i.}

Conjugation of Intermediates III-X and III-Y is achieved by coupling commercially available (Glen) Asymmetric Doubler Phosphoramidite, Intermediate V, to the 5′-OH of the desired Bᵢ that is 3′-attached to CPG using vendor-recommended procedures. Orthogonal Fmoc and DMT protecting groups in the Asymmetric Doubler Phosphoramidite thus allow detritylation (-DMT) of only one Linker component for subsequent coupling with commercially available (Glen) 5′-Carboxy-Modifier C10. As shown in FIG. 11, the resultant NHS moiety in the Fmoc-protected intermediate is then reacted with Intermediate III-X (see above) using conventional procedures. Subsequent serial reactions with diaza(1,3)bicyclo[5.4.0]undecane (DBU) to remove the Fmoc (-Fmoc) moiety and then coupling with 5′-Carboxy-Modifier C10 provides an NHS moiety for conjugation with Intermediate III-Y (see above) using conventional procedures. Cleavage from CPG and deprotection of DNA affords final product Structure 3.

11.4: One Primer Conjugated to Two Different Molecular Beacon-Type Probes Labeled with 3′-FAM/5′-Dabsyl and 3′-HEX/5′-Dabsyl

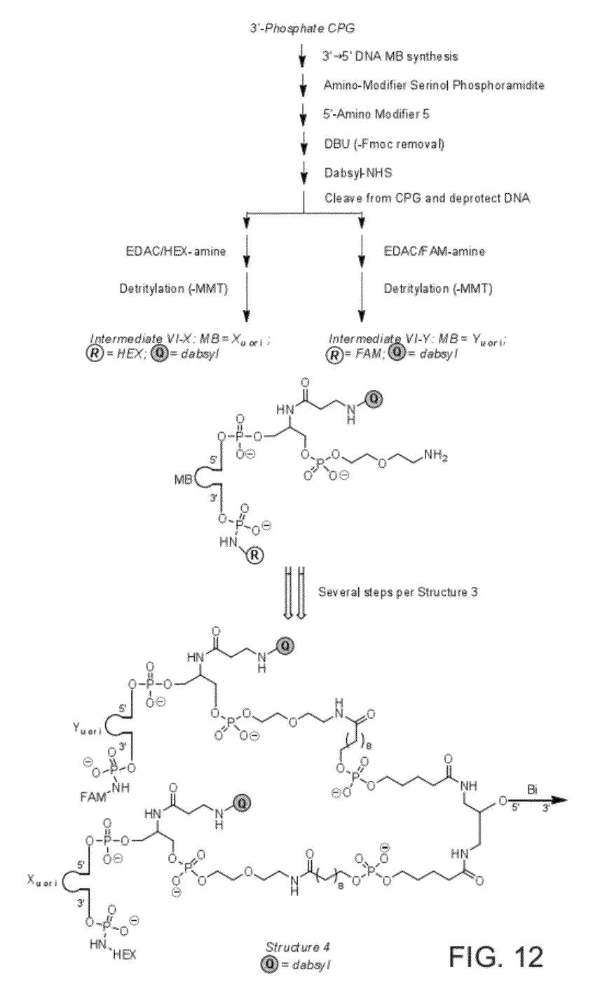

As shown in FIG. 12, commercially available (Glen) 3′-Phosphate CPG is used for conventional 3′→5′ solid-phase DNA synthesis of a desired first-MB sequence (X_{u or i}) followed by sequential (1.) coupling with commercially available Amino-Modifier Serinol Phosphoramidite (Glen), (2.) coupling with 5′-Amino-Modifier 5 (Glen), and (3.) reaction with diaza(1,3)bicyclo[5.4.0]undecane (DBU) to remove the Fmoc (-Fmoc) protecting group for coupling with Dabsyl-NHS (AnaSpec), all according to vendor-recommended procedures. These synthetic steps are repeated for a desired second-MB sequence (Y_{u or i}). Resultant first-MB and second-MB synthetic intermediates are each separately cleaved from CPG, DNA-deprotected, coupled with EDAC/HEX-amine (AnaSpec) or EDAC/FAM-amine (AnaSpec), respectively, according to vendor-recommended procedures, and then carefully detritylated (-MMT) in a polar non-aqueous/non-nucleophilic solvent (e.g., dimethylsulfoxide or formamide) to preclude 3′-phosphoramidate hydrolysis and thus provide Intermediate VI-X; R(2)=HEX; X_{u or i} and Intermediate VI-Y; R(1)=FAM; Y_{u or i}. Several steps (as illustrated in Sec. 9.3) are then carried out to afford final product Structure 4, which is the same as Structure 3 (see Sec. 9. 3) but with 3′-HEX/FAM and 5′-Dabsyl.

Although the present invention has been described in detail with reference to specific embodiments, those of skill in the art will recognize that modifications and improvements are within the scope and spirit of the invention, as set forth in the claims which follow. All publications and patent documents cited herein are incorporated herein by reference as if each such publication or document was specifically and individually indicated to be incorporated herein by reference. Citation of publications and patent documents (patents, published patent applications, and unpublished patent applications) is not intended as an admission that any such document is pertinent prior art, nor does it constitute any admission as to the contents or date of the same. The invention having now been described by way of written description, those of skill in the art will recognize that the invention can be practiced in a variety of embodiments and that the foregoing description is for purposes of illustration and not limitation of the following claims.

1. A multifunctional molecular probe comprising
a) a first oligonucleotide that is a primer with an extendible 3′ terminus,
b) a second oligonucleotide, comprising a first signal moiety,
wherein the first oligonucleotide and the second oligonucleotide are connected by a linker in a 5′-5′ orientation.

2. The multifunctional molecular probe of claim 1 wherein the 5′ terminus of the first oligonucleotide is connected by a linker to the 5′ terminus of the second oligonucleotide.

3. The multifunctional molecular probe of claim 1 wherein the first signal moiety comprises a first fluorophore and the first oligonucleotide does not comprise a signal moiety.

4. The multifunctional molecular probe of claim 3 wherein the second oligonucleotide comprises a second fluorophore, and the first and second fluorophores comprise a first donor-acceptor pair.

5. The multifunctional molecular probe of claim 1 comprising a third oligonucleotide,
wherein the first oligonucleotide and the third oligonucleotide are connected by a linker in a 5′-5′ orientation,
wherein the third oligonucleotide comprises a second signal moiety, which is different from the first signal moiety
wherein the first and second oligonucleotides and the first and third oligonucleotides may be linked to the first oligonucleotide by the same linker molecule or a different linker molecule.

6. The multifunctional molecular probe of claim 1 wherein the 5′ terminus of the third oligonucleotide is connected by a linker to the 5′ terminus of the first oligonucleotide.

7-9. (canceled)

10. The multifunctional molecular probe of claims 1-10 wherein
the second oligonucleotide comprises a donor fluorophore at or close to the 5′ terminus and an acceptor fluorophore at or close to the 3′ terminus, and
the second oligonucleotide comprises a partially self-complementary sequence such that the oligonucleotide may adopt a stem-and-loop structure in which the donor and acceptor are in close proximity or a linear structure in which the donor and acceptor are not in close proximity.

11-13. (canceled)

14. A molecular construct comprising two polynucleotides and a non-nucleotide linker, wherein
the first polynucleotide is an oligonucleotide comprising a signal moiety and comprising a sequence π;
the second polynucleotide comprises a target sequence and a probe binding sequence P, the target sequence being 5′ to P;
wherein π and P are sufficiently complementary to each other to hybridize and form a double stranded polynucleotide segment; and
wherein the linker links first and second polynucleotides in a 5′-5′ orientation.

15. The molecular construct of claim 14, wherein π and P are exactly complementary.

16. The molecular construct of claim 14, wherein the signal moiety comprises a fluorophore.

17. The molecular construct of claim 16 wherein signal moiety comprises a donor-acceptor pair.

18. The molecular construct of claim 14 wherein the oligonucleotide comprises a partially self-complementary sequence such that the oligonucleotide may adopt a stem-and-loop structure.

19-21. (canceled)

22. A pair of molecular constructs, each comprising three linked polynucleotides, wherein
1) the first molecular construct comprises
a first polynucleotide that is an oligonucleotide comprising a sequence π₁ and a first signal moiety;
a second polynucleotide that is an oligonucleotide comprising a sequence π₂ and a second signal moiety;
a third polynucleotide that comprises
a first target sequence, and
a probe binding sequence P₁, the target sequence being 5′ to P₁;
2) the second molecular constructs comprises
a second polynucleotide that is an oligonucleotide comprising a sequence π₁ and the first signal moiety;
a second polynucleotide that is an oligonucleotide comprising a sequence π₁ and the second signal moiety;
a third polynucleotide that comprises
a second target sequence, and
a probe binding sequence P₂, the target sequence being 5′ to P₂;
wherein in the first molecular construct, P₁ is sufficiently complementary to π₁ to hybridize and form a double stranded polynucleotide segment; and
wherein in the second molecular construct, P₂ is sufficiently complementary to π₂ to hybridize and form a double stranded polynucleotide segment; and
wherein the first and second target sequences are different
wherein the first and second signal moieties are different
wherein probe binding sequences P₁ and P₂ are different; and
wherein in each construct a linker links the first polynucleotide and the third polynucleotide in a 5′-5′ orientation and the same or a different linker molecule links the first polynucleotide and the third polynucleotide in a 5′-5′ orientation.

23. The pair of molecular constructs of claim 22, wherein π₁ and P₁ are exactly complementary and/or wherein π₂ and P₂ are exactly complementary.

24-26. (canceled)

27. The pair of molecular constructs of claim 22, wherein in one or both constructs in a pair, at least one of the first and second polynucleotides comprises a self-complementary (stem-loop forming) sequence.

28. The pair of molecular constructs of claim 27 wherein the first and second polynucleotides of both constructs comprise self-complementary (stem-loop forming) sequences.

29-31. (canceled)

32. A detection method comprising
i) combining
a) a molecular construct according to claim 14, wherein the second polynucleotide comprises an indexing sequence I′ and said indexing sequence is 3′ to, and optionally contiguous with, P;
b) an oligonucleotide primer comprising a sequence complementary to the indexing sequence; and
c) a DNA polymerase; and
ii) maintaining the combination under conditions in which the oligonucleotide primer is extended using the second polynucleotide as a template to produce an extension product, and the extension results in cleavage of the first polynucleotide and release of a fluorophore from the first polynucleotide; and
iii) detecting the release of the fluorophore.

33-35. (canceled)

36. A detection method comprising
i) amplifying a target sequence to produce a linear double-stranded amplicon, using primers comprising a first indexing sequence or its complement and a second indexing sequence or its complement, whereby the amplicon comprises the first indexing sequence at one end and the second indexing sequence at the other end;
ii) denaturing the double-stranded amplicon and carrying out two or more rounds of amplification, using as primers
1) a multifunctional molecular probe comprising
a) a first oligonucleotide that is a primer with an extendible 3′ terminus, wherein the primer comprises the first indexing sequence; and
b) a second oligonucleotide,
comprising a sequence π
comprising a donor-acceptor pair comprising a first member at or close to the 5′ terminus of the second oligonucleotide and an second member at or close to the 3′ terminus of the oligonucleotide; and
comprising a partially self-complementary sequence such that the oligonucleotide may adopt a stem-and-loop structure in which the donor and acceptor are in close proximity or a linear structure in which the donor and acceptor are not in close proximity;
wherein the first oligonucleotide and the second oligonucleotide are connected by a linker in a 5′-5′ orientation; and
2) a primer comprising
a) the second indexing sequence and
b) a probe binding sequence P positioned 5′ to the second indexing sequence;
wherein the amplification rounds produce a molecular construct of claim 14;
maintaining said molecular construct under conditions in which sequence π and sequence P hybridize and form a double stranded polynucleotide segment; whereby the second oligonucleotide adopts a linear structure in which the donor and acceptor are not in close proximity and produce a signal;
iii) detecting the signal.

37. The method of claim 36 wherein the 5′ terminus of the first oligonucleotide is connected by a linker to the 5′ terminus of the second oligonucleotide.

38. A detection method comprising
i) amplifying a target sequence to produce a linear double-stranded amplicon, using primers comprising a first indexing sequence or its complement and a second indexing sequence or its complement, whereby the double-stranded amplicon comprises the first indexing sequence at one end and the second indexing sequence at the other end;
ii) denaturing the double-stranded amplicon and carrying out two or more rounds of amplification, using as primers
1) a multifunctional molecular probe comprising
a) a first oligonucleotide that is a primer with an extendible 3′ terminus, wherein the primer comprises the first indexing sequence; and
b) a second oligonucleotide,
comprising a sequence π
comprising a donor-acceptor pair; and
wherein the first oligonucleotide and the second oligonucleotide are connected by a linker in a 5′-5′ orientation; and
2) a primer comprising the second indexing sequence,
wherein the amplification rounds produce a molecular construct of claim 14 comprising a first polynucleotide comprising a signal moiety and comprising a sequence π and a second polynucleotide comprising a target sequence and a probe binding sequence P, the target sequence being 5′ to P;
iii) maintaining said molecular construct in the presence of (a) an oligonucleotide primer comprising the sequence of the second indexing sequence and (b) DNA polymerase
under conditions in which sequence π and sequence P hybridize and form a double stranded polynucleotide segment; the oligonucleotide primer hybridizes to the second indexing sequence and is extended by the DNA polymerase using the second polynucleotide as a template to produce an extension product, and the extension results in cleavage of the first polynucleotide and release of a fluorophore from the first polynucleotide; and
iii) detecting the release of the fluorophore.

39-48. (canceled)
